(19) United States
(12) Patent Application Publication
(10) Pub. No.: US 2016/0184421 A1
(43) Pub. Date: Jun. 30, 2016

(54) IMMUNOTHERAPEUTIC TARGETS AGAINST STAPHYLOCOCCUS AUREUS

(71) Applicant: The University of Hong Kong, Hong Kong (CN)

(72) Inventors: Jian-Dong Huang, Hong Kong (CN); Kwok-Yung Yuen, Hong Kong (CN); Baozhong Zhang, Hong Kong (CN)

(21) Appl. No.: 14/974,964

(22) Filed: Dec. 18, 2015

Related U.S. Application Data
(60) Provisional application No. 62/093,752, filed on Dec. 18, 2014.

(51) Int. Cl. A61K 39/085; C07K 16/12

(52) U.S. Cl. CPC A61K 39/085; C07K 16/1271; A61K 2039/55505

(57) ABSTRACT

The subject invention pertains to vaccine formulations and antibodies, and related methods, for the treatment and/or prevention of S. aureus infection. The present invention provides one or more S. aureus antigens for use in vaccine formulations, wherein two or more antigens act synergistically. Further, the present invention provides vaccines that can protect against hematic spread, pneumonia and skin infection.

### CROSS-REFERENCE TO RELATED APPLICATION

This application claims the benefit of U.S. provisional application Ser. No. 62/093,752, filed Dec. 18, 2014, which is herein incorporated by reference in its entirety.

### BACKGROUND OF THE INVENTION

Staphylococcus aureus (S. aureus) is a facultative anaerobic Gram-positive bacterium, frequently found as part of the normal flora on the skin and in the nasal passages [1, 2]. S. aureus can cause a range of illnesses ranging from minor skin infections (such as pimples, cellulitis folliculitis, carbuncles, scalded skin syndrome and abscesses) to life-threatening diseases (such as meningitis, pneumonia, toxic shock syndrome, bacteraemia and sepsis). Multidrug-resistant (MDR) pathogens are a global problem. Their ability to adapt enables emerging strains to develop resistance to new antibiotics. Vaccinations could be a better strategy to control MDR pathogen infections. Vaccination has been demonstrated to be effective in preventing many infectious diseases, including influenza, small pox and Hepatitis B Virus infections. However, for many MDR pathogens, a serotype-independent immune response may be required.

Similar ESAT-6-like proteins, esxA (Rv3875) and esxB (Rv3874), secreted by M. tuberculosis are known to play a vital role in its pathogenesis. These two proteins can trigger cell-mediated immune responses and IFN-γ production during tuberculosis [46, 39]. This study indicated that the ESAT-6 expressed by the virulent M. tuberculosis strain H37Rv, but not by BCG, promotes immunity by enhancing Th17 cell responses [40]. Activation of naïve T cells by pathogen antigens presented by antigen presenting cells in the presence of various cytokines leads to the generation of T helper cell-subsets such as Th1, Th2 and Th17. Universally, the Th1 cells regulate IFN-γ-dependent immunity against most intracellular pathogens. The Th1 subset could be inhibited by IL-4, subsequently inducing another T cell subset, Th2, which produced IL-4, IL-5, and IL-13 against helminth infection [41].

A study by Misstear et al. showed that mouse nasal vaccination with targeted nanoparticles loaded with S. aureus protein could protect against systemic S. aureus infection in the absence of any antigen-specific antibodies [42]. This study suggested that a cellular-only response could protect against S. aureus infection. Moreover, many reports show the importance of Th17/IL-17 in the protection against S. aureus infections [43, 44]. Many mucosal vaccination approaches can induce robust Th17 responses, suggesting Th17 cells are useful targets for vaccines that induce immunity [45]. Recently, several studies using mouse vaccine models showed that T helper cells, including Th17, were important for a CD4⁺ T-cell-dependent immune response [45]. Th17 cells had a role in anti-microbial immunity at the epithelial/mucosal barrier [45]. Th17 cells produce cytokines, which stimulated epithelial cells to produce anti-microbial proteins to clear out certain types of opportunistic microbes.

Th17-mediated protective responses involve the release of anti-microbial peptides, recruitment of neutrophils, and IL-17-driven Th1 immunity. These signaling mechanisms could offer immunity against a range of MDR pathogens through the production and induction of inflammatory cytokines and other proteins. For staphylococcal vaccines to be effective, protection must be achieved against a wide variety of different clinical strains.

In the past, S. aureus infections were efficiently treated with antibiotics. However, the number of antibiotic resistant strains of S. aureus has been increasing in the past two decades. Most notably, Methicillin-resistant S. aureus (MRSA) is one of the most dangerous bacterial strains that have become resistant to many antibiotics. MRSA strains are very common in hospitals, but are also becoming increasingly prevalent in community-acquired infections [3, 4]. Hence, the development of immunotherapeutic approaches, either active or passive, has seen resurgence in recent years [5].

Previous studies show that S. aureus has many surface proteins and virulence factors, many of which have been evaluated as potential vaccine targets [6-15]. Past and present S. aureus vaccines or therapeutic antibody strategies mainly focus on the following targets: capsular polysaccharide, virulence factors, surface targets and iron-regulated proteins. The capsular polysaccharide is a putative protective antigen to develop as a S. aureus vaccine. The leading effort in this regard was StaphVAX, a bivalent polysaccharide and protein conjugated vaccine [16-19]. Some other strategies to develop a S. aureus vaccine have been based on virulence factors and surface proteins. Many virulence factors and surface proteins have been targeted by vaccination, including alpha-toxin (using non-toxic derivative H35L) [7, 20], clumping factor A (ClfA) [21], Fibronectin binding protein (FnBPA or FnBPB), Panton-Valentine leukocidin (PVL) [22] and protein A [11].

Another approach to develop a S. aureus vaccine has been based on iron-regulated proteins. Iron-regulated proteins are of fundamental importance to all bacterial pathogens (except Borrelia burgdorferi). The leading vaccine candidate in this regard was Merck V710, which is based on the S. aureus iron-regulated protein (IsdB) [6, 23]. The Merck V710 vaccine may be effective against hematic spread of the S. aureus infection, but may be ineffective against pneumonia and may not elicit any antibody opsonic activity.

The Sta-Ag1 protein is a cell wall-anchored enzyme, and acts as an immune evasion factor [29]. When both wild-type and AdsA mutant Staphylococci are mixed with fresh mouse or human blood, they are phagocytized by polymorphonuclear leukocytes (PMNs), particularly phagocytic neutrophils; however, wild-type Staphylococci survive within PMNs, but AdsA mutants are killed [29]. S. aureus can generate adenosine by converting AMP or ADP after infecting humans or mammals. In mammals, it is a two-step process to catalyze adenosine triphosphate to adenosine. First, ectonucleoside triphosphate diphosphohydrolases (ecto-NTDPases) hydrolyze ATP or ADP to produce AMP. AdsA contains two 5′-nucleotidase signature regions, which then catalyze the conversion of AMP to adenosine [30]. Extracellular adenosine is necessary for the regulation of inflammation, but excess production of adenosine is also harmful as in S. aureus infections [29]. S. aureus AdsA produces excessive adenosine, which disrupts the balance of the proinflammatory and anti-inflammatory response. Staphylococci survival in PMNs depends on adenosine receptor-mediated signaling. In addition, adenosine may also suppress adaptive immune responses by interfering with the antigen presenting cells (APCs) presenting S. aureus antigens [31].

Two typical S. aureus strains are Newman and USA 300. Newman is a methicillin-sensitive S. aureus strain and USA 300 is a community-associated methicillin-resistant S. aureus strain. The ‘Sta-Ag1’ antigen is annotated as ‘Adenosine synthase A (AdsA)’, similar to 5′-nucleotidase family protein. In the Newman strain, Sta-Ag1 is designated NWMN_0022 and has an amino acid sequence of GI:150373034, GenBank: BAF66294.1.

The ‘Sta-Ag2’ antigen is annotated as ‘Virulence factor SaEsxA’ and belongs to the ESAT-6 (esx) family. In the Newman strain, Sta-Ag2 is NWMN_0219 and has the amino acid sequence of GI: 68565377, UniProtKB/Swiss-Prot: P0C046.1.

The ‘Sta-Ag3’ antigen is annotated as ‘Virulence factor SaEsxB’ and belongs to the ESAT-6 (esx) family. In the Newman strain, Sta-Ag3 is NWMN_0225 and has the amino acid sequence of GI: 166214927, UniProtKB/Swiss-Prot: P0C047.2. These two ESAT-6-like proteins, SaEsxA and SaEsxB of S. aureus are secreted by a specialized secretion system termed ESAT-6-like system and play an important role in virulence. Mutants that failed to secrete EsxA and EsxB are defective to cause S. aureus-induced murine abscesses [32].

The ‘Sta-Ag4’ antigen is annotated as ‘Virulence factor SaEsxC’ and belongs to the ESAT-6 (esx) family. In the Newman strain, Sta-Ag4 is NWMN_0224 and has the amino acid sequence of SEQ ID NO:4 (GI:446933033, UniProtKB/Swiss-Prot: P0C051).

The ‘Sta-Ag5’ antigen is annotated as ‘phenol-soluble modulin alpha 1 (PSMal)’. In the Newman strain, Sta-Ag5 is NWMN_2619 and has an amino acid sequence of GI: 223670821, GenBank: AP009351.1. In the Newman strain, Sta-Ag5 is NWMN_2618 and has an amino acid sequence of.

The ‘Sta-Ag6’ antigen is annotated as ‘phenol-soluble modulin alpha 2 (PSMα2)’. In the Newman strain, Sta-Ag6 is NWMN_2618 and has an amino acid sequence of GI: 223670820, GenBank: AP009351.1

The ‘Sta-Ag7’ antigen is annotated as ‘phenol-soluble modulin alpha 3 (PSMα3)’. In the Newman strain, Sta-Ag7 is NWMN_2617 and has an amino acid sequence of GI: 223670819, GenBank: AP009351.1.

The ‘Sta-Ag8’ antigen is annotated as ‘phenol-soluble modulin alpha 4 (PSMα4)’. In the Newman strain, Sta-Ag8 is NWMN_2616 and has an amino acid sequence of GI: 223670818, GenBank: AP009351.1.

The ‘Sta-Ag9’ antigen is annotated as ‘Pmt A’, which is an ABC transporter, ATP-binding protein. In the Newman strain, Sta-Ag9 is NWMN_1869 and has an amino acid sequence of GI: 150374881, GenBank: AP009351.1.

The ‘Sta-Ag10’ antigen is annotated as ‘Pmt B’, which is an ABC transporter, ATP-binding protein. In the Newman strain, Sta-Ag10 is NWMN_1868 and has an amino acid sequence of GI: 150374880, GenBank: AP009351.1.

The ‘Sta-Ag11’ antigen is annotated as ‘Pmt C’, which is an ABC transporter, ATP-binding protein. In the Newman strain, Sta-Ag11 is NWMN_1867 and has an amino acid sequence of GI: 150374879, GenBank: AP009351.1.

The ‘Sta-Ag12’ antigen is annotated as ‘Pmt D’, which is an ABC transporter, ATP-binding protein. In the Newman strain, Sta-Ag12 is NWMN_1866 and has an amino acid sequence of GI: 150374878, GenBank: AP009351.1.

Sta-Ag1, Sta-Ag2, Sta-Ag3, Sta-Ag4, Sta-Ag5, Sta-Ag6, Sta-Ag7, Sta-Ag8, Sta-Ag9, Sta-Ag10, Sta-Ag11 and Sta-Ag12 represent desirable antigens for vaccine development.

In terms of passive immunization, most strategies are aimed to eliminate major S. aureus virulence determinants such as monoclonal alpha-toxin antibodies, polyclonal PVL antibodies and anti-ClfA monoclonal antibody (Aurexis). However, thus far, most of the clinical trials for S. aureus vaccines or passive immunization have ended in failure (Nabi, Types 5 and 8 CPS conjugated to pseudomonal exoprotein A [19]; Inhibitex, ClfA, SdrG (Veronate) [24, 25]; Nabi, Polyclonal anti-CPS 5 and 8 (Altastaph) [26, 27]). The reasons why the S. aureus vaccine clinical trials may have failed were analyzed by Protor [28].

### BRIEF SUMMARY OF THE INVENTION

The present invention provides vaccine formulations and antibodies, and related methods, for the treatment and/or prevention of S. aureus infection. The present invention provides one or more S. aureus antigens for use in vaccine formulations, wherein two or more antigens act synergistically. Thus, the protection against S. aureus infection achieved by their combined administration exceeds that expected by mere addition of their individual protective efficacy. Further, the present invention provides vaccines that can protect against hematic spread, pneumonia and skin infection, and which may also elicit a protective antibody response. The invention also provides novel antibodies and antibody cocktails to treat S. aureus infection. Furthermore, the vaccine formulations and antibodies of the present invention can be utilized in the treatment of mastitis in lactating dairy cows caused by S. aureus infection.

In one aspect, the present invention provides a vaccine formulation comprising one or more S. aureus polypeptides, as well as fragments, variants or derivatives thereof. The S. aureus polypeptide is selected from Sta-Ag1, Sta-Ag2, Sta-Ag3, Sta-Ag4, Sta-Ag5, Sta-Ag6, Sta-Ag7, Sta-Ag8, Sta-Ag9, Sta-Ag10, Sta-Ag1, Sta-Ag12, and combinations thereof.

In some embodiments, the formulations may further include one or more adjuvants, such as, for example, a Th1 adjuvant, a Th2 adjuvant, a Th17 adjuvant, an aluminum hydroxide adjuvant, or combinations thereof. In additional embodiments, the formulation may include a histidine buffer.

In some embodiments, the S. aureus polypeptide is derived from a eukaryotic expression system. In other embodiments, the S. aureus polypeptide is derived from a prokaryotic expression system. In certain embodiments, the S. aureus polypeptide may be a synthetic polypeptide.

The Sta-Ag1 S. aureus polypeptides of the present invention may comprise the amino acid sequence of SEQ ID NO: 1, or a bioequivalent fragment, variant or derivative thereof. The Sta-Ag2 S. aureus polypeptides of the present invention may comprise the amino acid sequence of SEQ ID NO: 2, or a fragment, variant or derivative thereof. The Sta-Ag3 S. aureus polypeptides of the present invention may comprise the amino acid sequence of SEQ ID NO: 3, or a bioequivalent fragment, variant or derivative thereof. The Sta-Ag4 S. aureus polypeptides of the present invention may comprise the amino acid sequence of SEQ ID NO: 4, or a bioequivalent fragment, variant or derivative thereof. The Sta-Ag5 S. aureus polypeptides of the present invention may comprise the amino acid sequence of SEQ ID NO: 5, or a bioequivalent fragment, variant or derivative thereof. The Sta-Ag6 S. aureus polypeptides of the present invention may comprise the amino acid sequence of SEQ ID NO: 6, or a bioequivalent fragment, variant or derivative thereof. The Sta-Ag7 S. aureus polypeptides of the present invention may comprise the amino acid sequence of SEQ ID NO: 7, or a bioequivalent fragment, variant or derivative thereof. The Sta-Ag8 S. aureus polypeptides of the present invention may comprise the amino acid sequence of SEQ ID NO: 8, or a bioequivalent fragment, variant or derivative thereof. The Sta-Ag9 S. aureus polypeptides of the present invention may comprise the amino acid sequence of SEQ ID NO: 9, or a bioequivalent fragment, variant or derivative thereof. The Sta-Ag10 S. aureus polypeptides of the present invention may comprise the amino acid sequence of SEQ ID NO: 10, or a fragment, variant or derivative thereof. The Sta-Ag11 S. aureus polypeptides of the present invention may comprise the amino acid sequence of SEQ ID NO: 11, or a fragment, variant or derivative thereof. The Sta-Ag12 S. aureus polypeptides of the present invention may comprise the amino acid sequence of SEQ ID NO: 12, or a fragment, variant or derivative thereof.

In preferred embodiments, the present invention provides combinations of Sta-Ags based on SEQ ID NOs: 1 to 12, which combinations exert synergistic effects in eliciting a S. aureus-specific immune response.

In another aspect, the present invention provides isolated antibodies that bind to at least one of Sta-Ag1, Sta-Ag2, Sta-Ag3, Sta-Ag4, Sta-Ag5, Sta-Ag6, Sta-Ag7, Sta-Ag8, Sta-Ag9, Sta-Ag10, Sta-Ag11 and/or Sta-Ag12 S. aureus polypeptides, or bioequivalent fragments, variants, or derivatives thereof.

In another aspect, the present invention provides methods for preventing and/or treating S. aureus infection in a subject, comprising administering to the subject an effective amount of a vaccine formulation comprising one or more S. aureus polypeptides, or bioequivalent fragments, variants or derivatives thereof. The S. aureus polypeptide is selected from Sta-Ag1, Sta-Ag2, Sta-Ag3, Sta-Ag4, Sta-Ag5, Sta-Ag6, Sta-Ag7, Sta-Ag8, Sta-Ag9, Sta-Ag10, Sta-Ag11, Sta-Ag12, and combinations thereof.

In yet another aspect, the present invention provides methods for preventing and/or treating S. aureus infection in a subject comprising administering to the subject an effective amount of one or more antibodies selected from a Sta-Ag1 antibody, a Sta-Ag2 antibody, a Sta-Ag3 antibody, a Sta-Ag4 antibody, a Sta-Ag5 antibody, a Sta-Ag6 antibody, a Sta-Ag7 antibody, a Sta-Ag8 antibody, a Sta-Ag9 antibody, a Sta-Ag10 antibody, Sta-Ag11, and a Sta-Ag12 antibody.

The methods and compositions herein described can be used in connection with pharmaceutical, medical, and veterinary applications, as well as fundamental scientific research and methodologies, as would be identifiable by a skilled person upon reading of the present disclosure. These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying figures described below.

FIG. 1 shows a model to generate protective immunity against S. aureus infections through vaccination (adapted from Nature Medicine 17, 168-169 (2011) doi: 10.1038/nm0211-168).

FIG. 2 shows evaluation of serum antibody responses in mice by ELISA. The results of two independent experiments are shown.

FIGS. 3A and 3B show graphs illustrating that active immunization with Sta-Ag1 decreases the size of abscesses caused by USA300 or Newman strains of S. aureus. Mice were injected intramuscularly with aluminium hydroxide gel (AHG) plus phosphate-buffered saline (PBS). A and B, Abscess formation was monitored once per day after subcutaneous infection with 1×10⁷ of the indicated bacteria 49 days after primary immunization. Results are the mean value±standard error of the mean; n=12 mice per group. *p<0.05 versus wild-type USA300 or Newman strains using a 2-way analysis of variance and Bonferroni's post-test.

FIGS. 4A, 4B, and 4C show the percentage of mice per group that had dermonecrosis on each day following active immunization with Sta-Ag1 and challenge with USA300 or Newman skin inoculation. A P value of P<0.001 was obtained for mock immunized mice after infection with either Newman (a) or USA300 (b) strains over the 14-day time course. C, shows representative mouse skin lesions on day 4. Red arrows indicate dermonecrosis, black arrows indicate abscess formation without dermonecrosis, and yellow boxes indicate scratches.

FIG. 5 shows a graph of the survival curve of vaccinated BALB/c mice challenged with S. aureus. Mice were challenged by intravenous injection of S. aureus ATCC 25923 (5×10⁷ CFU). The results of two independent experiments (mice, n=12) are shown. The p value represents the likelihood of a significant difference between all groups by pair-wise log-rank analysis.

FIG. 6 shows a graph of the evaluation of the therapeutic effects of anti-Sta-Ag1 rabbit serum in the BALB/c mouse model. Mice were challenged by intravenous injection of S. aureus ATCC 25923 (5×10⁷ CFU). After 2 hours, the experimental groups were treated with anti-Sta-Ag1 rabbit serum, whereas control mice were injected with normal rabbit serum. The p value represents the likelihood of a significant difference between all groups by pair-wise log-rank analysis.

FIG. 7 shows a graph of the evaluation of the therapeutic effects of anti-AdsA (anti-Sta-Ag1) mouse serum in the BALB/c mouse model. Mice were challenged by intravenous injection of S. aureus ATCC 25923 (5×10⁷ CFU). After 2 hours, the experimental groups were treated with anti-AdsA mouse serum, whereas control mice were injected with normal mouse serum. The p value represents the likelihood of a significant difference between all groups by pair-wise log-rank analysis.

FIGS. 8A and 8B show that passive immunization with AdsA-specific (Sta-Ag1-specific) mouse anti-sera prevents dermonecrosis. FIG. 8A, Percentage of mice per group that had dermonecrosis on each day. *P<0.001 for mice administered pre-immune versus anti-AdsA serum after infection with ATCC25923 strains over the 14-day time course. FIG. 8B, Representative skin lesions of mice on day 3 for each of the treatment conditions anti-AdsA, AdsA-specific mouse anti-sera; Pre-immune, pre-immune mouse serum samples. Red arrows indicate dermonecrosis, and black arrows indicate abscess formation without dermonecrosis.

FIGS. 9A and 9B show graphs illustrating that passive immunization with Sta-Ag1-specific rabbit anti-sera (anti-Sta-Ag1) reduces size of lesions caused by USA300 or Newman strains of S. aureus. a and b, Mice received 100 mL of pre-immune rabbit serum samples (pre-immune) or anti-Sta-Ag14 h before subcutaneous infection with 1×10⁷ with USA300 or Newman strains and on day 2 after infection. Results are the mean value±standard error of the mean for all groups; n=10 mice per group. *p<0.05 versus wild-type USA300 or Newman strains using a 2-way analysis of variance and Bonferroni's post-test.

FIGS. 10A and 10B shows graphs illustrating that passive immunization with Sta-Ag1-specific rabbit anti-sera (anti-Sta-Ag1) prevents dermonecrosis. *p<0.001 for mice administered pre-immune versus anti-Sta-Ag1 serum after infection with Newman (a) or USA300 (b) strains over the 14-day time course.

FIGS. 11A, 11B, 11C, 11D, 11E, 11F, 11G, 11H, 11I shows that immunization with the rSaEsxA, rSaEsxB and rSaEsxA+B generates protective immunity against S. aureus abscess formation in BALB/C mice. Animals were treated with PBS plus AHG (FIG. 11A, FIG. 11B) or immunized with rSaEsxA plus AHG (FIG. 11C, FIG. 11D), rSaEsxB (FIG. 11E, FIG. 11F) and rSaEsxA+B plus AHG (FIG. 11G, FIG. 11I); and challenged by intraperitoneal infection with S. aureus ATCC 25923. Four days after challenge, mice were killed, and the kidneys were collected for histopathology (A-H) or Staphylococcal load measurements (FIG. 11I). Kidney was fixed with formalin, thin-sectioned, and stained with hematoxylin/eosin. Microscopic images of whole kidneys (FIG. 11A, FIG. 11C, FIG. 11E and FIG. 11G) or tissue at magnification (FIG. 11B, FIG. 11D, FIG. 11F and FIG. 11H) revealed abscess formation only in PBS control mice. Consistent results were obtained for six kidney tissues in each group. Staphylococcal abscess (black arrow) with a central concentration of staphylococci (red arrow) was marked in FIG. 11B. Small infiltrates of PMNs (black arrow) were shown in FIG. 11D, FIG. 11F and FIG. 11H. (Scale bars: A, C, E and G, 1000 μm; B, D, E and F, 50 μm.)

FIG. 12 shows the survival curve of vaccinated BALB/C mice following S. aureus challenge. Mice were challenged by intravenously injection of S. aureus ATCC2593 (5×10⁷ CFU). P value represents the likelihood of a significant difference between all groups following pair-wise log-rank analysis between groups. “NS” indicates differences not significant; *p>0.05 following pair-wise log-rank analysis. The data are the results of three independent experiments.

FIG. 13 shows that immunization with Sta-Ag2+3 generates protective immunity against lethal challenge with two different clinical S. aureus strains. Mice (n=10) immunized with the Sta-Ag2+3 or treated with PBS+AHG as controls were challenged with S. aureus Newman or USA 300 strains by intravenous injection. The survival of mice was monitored for 14 days. Log-rank (Mantel-Cox) test was used to compare the protective immunity between control mice and the Sta-Ag2+3 immunized mice. Data from two replicate experiments is shown.

FIGS. 14A, 14B, and 14C show the antigen-specific IL17A and IFN-γ responses elicited by Sta-Ag2 or Sta-Ag3 immunization. Immunized mice (n=8) were sacrificed 5 days after the third immunization and splenocytes were prepared and stimulated with Sta-Ag2 or Sta-Ag3 protein for 20 h. Detection of (FIG. 14A) IL-17A and (FIG. 14B) IFN-γ producing cells by ELISPOT. (FIG. 14C) Representative images of splenic ELISPOT responses. Results for one of two representative experiments are shown. The Mann Whitney test was used for the statistical analysis. Data was expressed as mean+SEM. SFCs: spot-forming units; IMSA: immunized mice stimulated with Sta-Ag2; IMSB: immunized mice stimulated with Sta-Ag3; NMSA: naïve mice stimulated with Sta-Ag2; NMSB: naïve mice stimulated with Sta-Ag3; UNS: unstimulated wells (immunized mice).

FIG. 15 shows the survival curve of BALB/c mice passively treated with SaEsxA- or SaEsxB-specific mouse antisera and challenged with the Newman strain via the tail vein. BALB/c mice (n=10) received 100 μL of normal mouse serum or specific mouse antiserum (anti-SaEsxA or anti-SaEsxB) 4 hours before intravenous injection of S. aureus Newman strain (5×10⁷ CFU) and on day 2 after infection. Log-rank (Mantel-Cox) test was used to compare between groups. NS: no significant differences (p>0.05). Data from two replicate experiments is shown.

FIG. 16 shows evaluation of serum antibody responses in mice by ELISA. Sta-C3 (Sta-Ag2, Sta-Ag3 and Sta-Ag4), Sta-C4 (Sta-Ag-9, Sta-Ag-10, Sta-Ag-11 and Sta-Ag-12) and Sta-C9 (Sta-Ag1, Sta-Ag2, Sta-Ag3, Sta-Ag4, Sta-Ag-9, Sta-Ag-10, Sta-Ag-11, Sta-Ag-12 and uric acid).

FIG. 17 shows representative mouse skin lesions (day 4). Black arrows indicate dermonecrosis, and red arrows indicate abscess formation without dermonecrosis. Sta-C3 (Sta-Ag2, Sta-Ag3 and Sta-Ag4), Sta-C4 (Sta-Ag-9, Sta-Ag-10, Sta-Ag-11 and Sta-Ag-12) and Sta-C9 (Sta-Ag1, Sta-Ag2, Sta-Ag3, Sta-Ag4, Sta-Ag-9, Sta-Ag-10, Sta-Ag-11, Sta-Ag-12 and uric acid).

FIG. 18 shows percentage of mice per group that had dermonecrosis on each day. Sta-C3 (Sta-Ag2, Sta-Ag3 and Sta-Ag4), Sta-C4 (Sta-Ag-9, Sta-Ag-10, Sta-Ag-11 and Sta-Ag-12) and Sta-C9 (Sta-Ag1, Sta-Ag2, Sta-Ag3, Sta-Ag4, Sta-Ag-9, Sta-Ag-10, Sta-Ag-11, Sta-Ag-12 and uric acid).

FIGS. 19A and 19B shows that immunization with combined vaccine generates protective immunity against lethal challenge with S. aureus USA300 strains. The survival of mice was monitored for 14 days. Log-rank (Mantel-Cox) test was used to compare the protective immunity between different groups. Sta-C3 (Sta-Ag2, Sta-Ag3 and Sta-Ag4), Sta-C4 (Sta-Ag-9, Sta-Ag-10, Sta-Ag-11 and Sta-Ag-12) and Sta-C9 (Sta-Ag1, Sta-Ag2, Sta-Ag3, Sta-Ag4, Sta-Ag-9, Sta-Ag-10, Sta-Ag-11, Sta-Ag-12 and uric acid).

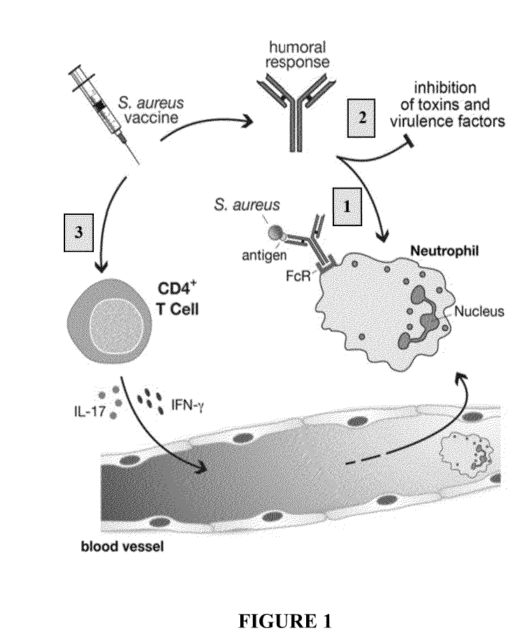

### BRIEF DESCRIPTION OF THE SEQUENCES

SEQ ID NO: 1 is an amino acid sequence of S. aureus Sta-Ag1.

SEQ ID NO: 2 is an amino acid sequence of S. aureus Sta-Ag2.

SEQ ID NO: 3 is an amino acid sequence of S. aureus Sta-Ag3.

SEQ ID NO: 4 is an amino acid sequence of S. aureus Sta-Ag4.

SEQ ID NO: 5 is an amino acid sequence of S. aureus Sta-Ag5.

SEQ ID NO: 6 is an amino acid sequence of S. aureus Sta-Ag6.

SEQ ID NO: 7 is an amino acid sequence of S. aureus Sta-Ag7.

SEQ ID NO: 8 is an amino acid sequence of S. aureus Sta-Ag8.

SEQ ID NO: 9 is an amino acid sequence of S. aureus Sta-Ag9.

SEQ ID NO: 10 is an amino acid sequence of S. aureus Sta-Ag10.

SEQ ID NO: 11 is an amino acid sequence of S. aureus Sta-Ag11.

SEQ ID NO: 12 is an amino acid sequence of S. aureus Sta-Ag12.

### DETAILED DISCLOSURE OF THE INVENTION

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Generally, vaccines or antibodies to S. aureus are based on a single antigen and do not provide sufficient protection against hematic spread, pneumonia and skin infection. Therefore, the selection of potent antigenic targets which induce protective immunity is crucial in the development of therapeutics based on vaccines and/or antibodies.

The subject invention provides vaccine formulations and antibodies, and related methods, for the treatment and/or prevention of S. aureus infection. In some embodiments, the subject invention provides one or more S. aureus antigens for use in vaccine formulations, wherein two or more antigens act synergistically. Thus, the protection against S. aureus infection achieved by their combined administration exceeds that expected by mere addition of their individual protective efficacy.

In preferred embodiments, the subject invention provides vaccines which can protect against hematic spread, pneumonia and skin infection, and which may also elicit a protective antibody response. In further preferred embodiments, the subject invention provides novel antibodies and antibody cocktails to treat S. aureus infection.

As used herein, the term “vaccine” or “immunizing formulation” refers to any composition that stimulates an immune response to a particular antigen or antigens. Thus, a vaccine refers to any composition that is administered to a subject with the goal of establishing an immune response and/or immune memory to a particular antigen. In some embodiments of the subject invention, the vaccine compositions comprise other substances designed to increase the ability of the vaccine to generate an immune response.

In preferred embodiments, the vaccines of the subject invention can be therapeutic or prophylactic. Thus, for example, the vaccines disclosed herein can be used to prevent an infection, such as S. aureus infection. Alternatively, the vaccines disclosed herein can be used therapeutically to treat a subject with a S. aureus infection.

In some embodiments, the disclosed methods of the subject invention comprise the simultaneous or separate administration of multiple vaccines or vaccine components. Thus, in further embodiments, the subject invention provides the administration of a second, third, fourth, etc. S. aureus polypeptide, wherein the second, third, fourth, etc. S. aureus polypeptide is administered in a separate vaccine for administration at the same time as or 1, 2, 3, 4, 5, 6, 10, 14, 18, 21, 30, 60, 90, 120, 180, 360 days (or any number of days in between) after the first S. aureus polypeptide.

In some embodiments, the subject invention provides a vaccine formulation comprising one or more S. aureus polypeptides, or bioequivalent fragments, variants or derivatives thereof. In preferred embodiments, the S. aureus polypeptide is one of Sta-Ag1, Sta-Ag2, Sta-Ag3, Sta-Ag4, Sta-Ag5, Sta-Ag6, Sta-Ag7, Sta-Ag8, Sta-Ag9, Sta-Ag10, Sta-Ag11, Sta-Ag12, and/or combinations thereof. Fragments, variants, and derivatives are routinely prepared by those of ordinary skill in the art and their immunogenicity is readily and routinely determined. Immunogenic fragments, variants and derivatives are equivalents (also known as “bioequivalents”) of these S. aureus polypeptides and are included in the scope of the subject invention.

In preferred embodiments, the subject invention provides vaccine formulations comprising the S. aureus polypeptides from Sta-Ag1, Sta-Ag2, and/or Sta-Ag3. In further preferred embodiments, the subject invention provides vaccine formulations comprising the S. aureus polypeptides from Sta-Ag2 combined with any of Sta-Ag1, Sta-Ag4, Sta-Ag5, Sta-Ag6, Sta-Ag7, Sta-Ag8, Sta-Ag9, Sta-Ag10, Sta-Ag11, and/or Sta-Ag12. In yet further preferred embodiments, the subject invention provides vaccine formulations comprising the S. aureus polypeptides from Sta-Ag3 combined with any of Sta-Ag1, Sta-Ag4, Sta-Ag5, Sta-Ag6, Sta-Ag7, Sta-Ag8, Sta-Ag9, Sta-Ag10, Sta-Ag11, and/or Sta-Ag12.

In further embodiments, the subject invention provides vaccine formulations comprising the S. aureus polypeptides from Sta-Ag1, Sta-Ag2, Sta-Ag3, Sta-Ag4, Sta-Ag5, Sta-Ag6, Sta-Ag7, Sta-Ag8, Sta-Ag9, Sta-Ag10, Sta-Ag11, and/or Sta-Ag12, and/or any combinations thereof.

In more preferred embodiments, the subject invention provides vaccine formulations comprising the S. aureus polypeptides from Sta-Ag2, Sta-Ag3, and Sta-Ag4, designated combination Sta-C3.

In other more preferred embodiments, the subject invention provides vaccine formulations comprising the S. aureus polypeptides from Sta-Ag9, Sta-Ag10, Sta-Ag11, and/or Sta-Ag12, designated combination Sta-C4.

In most preferred embodiments, the subject invention provides vaccine formulations comprising the S. aureus polypeptide from Sta-Ag1, Sta-Ag2, Sta-Ag3, Sta-Ag4, Sta-Ag9, Sta-Ag10, Sta-Ag1, and/or Sta-Ag12, designated combination Sta-C9.

In some embodiments, the formulations of the subject invention may further include one or more adjuvant, such as, for example, a Th1 adjuvant, a Th2 adjuvant, a Th17 adjuvant, an aluminum hydroxide adjuvant, or combinations thereof. In additional embodiments, the formulation may include a histidine buffer.

In some embodiments, the S. aureus polypeptide is derived from a eukaryotic expression system. In other embodiments, the S. aureus polypeptide is derived from a prokaryotic expression system. In certain embodiments, the S. aureus polypeptide may be a synthetic polypeptide or a recombinant polypeptide.

In preferred embodiments, the S. aureus polypeptides of the subject invention are derived from various strains of S. aureus bacteria, including, but not limited to, USA 300 and Newman strains. In further preferred embodiments, the polypeptides, fragments thereof, or antibodies are delivered to a subject by any means known in the art, including, but not limited to, Salmonella and virus-like particle (VLP) delivery systems.

In preferred embodiments of the subject invention, the Sta-Ag1 S. aureus polypeptides may comprise the amino acid sequence of SEQ ID NO: 1, or a fragment, variant or derivative thereof, which SEQ ID NO: 1 is derived from the Newman strain Sta-Ag1 designated NWMN_0022 and has an amino acid sequence of GI: 150373034, GenBank: BAF66294.1. In more preferred embodiments, fragments useful in the subject invention comprise amino acids from about amino acid 36 to about amino acid 430 of SEQ ID NO: 1, which fragments contain two 5′-nucleotidase motifs.

In other preferred embodiments of the subject invention, the Sta-Ag2 S. aureus polypeptides may comprise the amino acid sequence of SEQ ID NO: 2, or a bioequivalent fragment, variant or derivative thereof, which SEQ ID NO: 2 is derived from Newman strain Sta-Ag2 designated NWMN_0219 and has an amino acid sequence of GI: 68565377, UniProtKB/Swiss-Prot: P0C046.1.

In other preferred embodiments of the subject invention, the Sta-Ag3 S. aureus polypeptides of the present invention may comprise the amino acid sequence of SEQ ID NO: 3, or a bioequivalent fragment, variant or derivative thereof, which SEQ ID NO: 3 is derived from Newman strain Sta-Ag3 designated NWMN_0225 and has an amino acid sequence of GI: 166214927, UniProtKB/Swiss-Prot: P0C047.2.

In other preferred embodiments of the subject invention, the Sta-Ag4 S. aureus polypeptides of the present invention may comprise the amino acid sequence of SEQ ID NO: 4, or a bioequivalent fragment, variant or derivative thereof, which SEQ ID NO: 4 is derived from Newman strain Sta-Ag4 designated NWMN_0224 and has an amino acid sequence of GI:446933033, GenBank: AP009351.1.

In other preferred embodiments of the subject invention, the Sta-Ag5 S. aureus polypeptides of the present invention may comprise the amino acid sequence of SEQ ID NO: 5, or a bioequivalent fragment, variant or derivative thereof, which SEQ ID NO: 5 is derived from Newman strain Sta-Ag5 designated NWMN_2619 and has an amino acid sequence of GI: 223670821, GenBank: AP009351.1.

In other preferred embodiments of the subject invention, the Sta-Ag6 S. aureus polypeptides of the present invention may comprise the amino acid sequence of SEQ ID NO: 6, or a bioequivalent fragment, variant or derivative thereof, which SEQ ID NO: 6 is derived from Newman strain Sta-Ag6 designated NWMN_2618 and has an amino acid sequence of GI: 223670820, GenBank: AP009351.1.

In other preferred embodiments of the subject invention, the Sta-Ag7 S. aureus polypeptides of the present invention may comprise the amino acid sequence of SEQ ID NO: 7, or a bioequivalent fragment, variant or derivative thereof, which SEQ ID NO: 7 is derived from Newman strain Sta-Ag7 designated NWMN_2617 and has an amino acid sequence of GI: 223670819, GenBank: AP009351.1.

In other preferred embodiments of the subject invention, the Sta-Ag8 S. aureus polypeptides of the present invention may comprise the amino acid sequence of SEQ ID NO: 8, or a bioequivalent fragment, variant or derivative thereof, which SEQ ID NO: 8 is derived from Newman strain Sta-Ag8 designated NWMN_2616 and has an amino acid sequence of GI:223670818, GenBank: AP009351.1.

In other preferred embodiments of the subject invention, the Sta-Ag9 S. aureus polypeptides of the present invention may comprise the amino acid sequence of SEQ ID NO: 9, or a bioequivalent fragment, variant or derivative thereof, which SEQ ID NO: 9 is derived from Newman strain Sta-Ag9 designated NWMN_1869 and has an amino acid sequence of GI: 150374881, GenBank: AP009351.1.

In other preferred embodiments of the subject invention, the Sta-Ag10 S. aureus polypeptides of the present invention may comprise the amino acid sequence of SEQ ID NO: 10, or a bioequivalent fragment, variant or derivative thereof, which SEQ ID NO: 10 is derived from Newman strain Sta-Ag10 designated NWMN_1868 and has an amino acid sequence of SEQ ID NO: 10 (GI: 150374880, GenBank: AP009351.1).

In other preferred embodiments of the subject invention, the Sta-Ag11 S. aureus polypeptides of the present invention may comprise the amino acid sequence of SEQ ID NO: 11, or a bioequivalent fragment, variant or derivative thereof, which SEQ ID NO: 11 is derived from Newman strain Sta-Ag11 designated NWMN_1867 and has an amino acid sequence of GI: 150374879, GenBank: AP009351.1.

In other preferred embodiments of the subject invention, the Sta-Ag12 S. aureus polypeptides of the present invention may comprise the amino acid sequence of SEQ ID NO: 12, or a bioequivalent fragment, variant or derivative thereof, which SEQ ID NO: 12 is derived from Newman strain Sta-Ag12 designated NWMN_1866 and has an amino acid sequence of GI: 150374878, GenBank: AP009351.1.

In another preferred embodiment, the subject invention provides isolated antibodies or aptamers that bind to at least one of Sta-Ag1, Sta-Ag2, Sta-Ag3, Sta-Ag4, Sta-Ag5, Sta-Ag6, Sta-Ag7, Sta-Ag8, Sta-Ag9, Sta-Ag10, Sta-Ag11, and/or Sta-Ag12 S. aureus polypeptides, or bioequivalent fragments, variants, or derivatives thereof. Antibodies may include intact immunoglobulin molecules, as well as fragments thereof, which are capable of binding associated antigens of Sta-Ag1, Sta-Ag2, Sta-Ag3, Sta-Ag4, Sta-Ag5, Sta-Ag6, Sta-Ag7, Sta-Ag8, Sta-Ag9, Sta-Ag10, Sta-Ag11, and/or Sta-Ag12 and can include chimeric antibody molecules; F (ab′)₂ and F (ab) fragments and Fv molecules; non-covalent heterodimers; single chain Fv molecules (scFv); dimeric and trimeric antibody fragment constructs; minibodies and humanized antibody molecules.

In further embodiments, the subject invention provides a cocktail of antibodies which are specific for PSM antigens and includes any of PSMα1 (Sta-Ag5), PSMα2 (Sta-Ag6), PSMα3 (Sta-Ag7), and PSMα4 (Sta-Ag8) Advantageously, PSM antigens are secreted and crucial to S. aureus virulence, therefore targeting PSMs with monoclonal antibodies (mAbs) can provide enhanced protection

In another embodiment, the subject invention provides methods for preventing and/or treating S. aureus infection in a subject, comprising administering to the subject an effective amount of a vaccine formulation comprising one or more S. aureus polypeptides, or bioequivalent fragments, variants or derivatives thereof, wherein the S. aureus polypeptide is one of Sta-Ag1, Sta-Ag2, Sta-Ag3, Sta-Ag4, Sta-Ag5, Sta-Ag6, Sta-Ag7, Sta-Ag8, Sta-Ag9, Sta-Ag10, Sta-Ag1, Sta-Ag12, and/or any combination thereof.

As used herein, the term “subject” refers to an animal. Typically, the terms “subject” and “patient” may be used interchangeably herein in reference to a subject. As such, a “subject” includes an animal that is being treated for a disease, being immunized, or the recipient of a mixture of components as described herein, such as a vaccine formulation or antibody. The term “animal,” includes, but is not limited to, mouse, rat, dog, guinea pig, cow, horse, chicken, cat, rabbit, pig, monkey, chimpanzee, and human.

In yet another embodiment, the present invention provides methods for preventing or treating S. aureus infection in a subject in need thereof comprising administering to the subject an effective amount of one or more antibody which antibody can be a Sta-Ag1 antibody, a Sta-Ag2 antibody, a Sta-Ag3 antibody, a Sta-Ag4 antibody, a Sta-Ag5 antibody, a Sta-Ag6 antibody, a Sta-Ag7 antibody, a Sta-Ag8 antibody, a Sta-Ag9 antibody, a Sta-Ag10 antibody, a Sta-Ag11 or a Sta-Ag12 antibody, and/or any combination thereof.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Example 1

Animal model to evaluate the immunogenicity of S. aureus antigens. The S. aureus ATCC 25923, ATCC 29213, Newman and USA 300 strains were stored at −80° C. until use. E. coli strain BL21 (DE3) was used for protein expression. Recombinant expression vector pETH was obtained from Prof. K Y Yuen. SPF BALB/c mice were supplied by the Laboratory Animal Unit of the University of Hong Kong. All animal experiments were approved by the Committee on the Use of Live Animals in Teaching & Research of the University of Hong Kong (CULATR 2596-11).

Animal immunization. Six-week-old female BALB/c mice (n=6 per group) were immunized with a range of doses (Table 1) of rSaEsxA or rSaEsxB proteins with Freund's adjuvant by intraperitoneal (i.p.) injection or with Aluminium hydroxide gel (Alhydrogel, AHG) by intramuscular (i.m.) injection. The two recombinant proteins were emulsified at a ratio of 1:1 with complete Freund's adjuvant (CFA) for priming and with incomplete Freund's adjuvant (IFA) for boosting. The two recombinant proteins were formulated at a ratio of 9:1 with AHG (100 μl of 2% AHG per 900 μl of antigen). These treatments were administered to mice on days 0, 14, and 28. Blood samples were drawn from the tail vein on days 0, 21, and 35.

Antibody detection. The rSaEsxA and rSaEsxB antibody titers were detected by enzyme-linked immunosorbent assay (ELISA). Briefly, rSaEsxA or rSaEsxB protein (1 μg/ml in 0.05 M carbonate/bicarbonate buffer, pH 9.6) were coated (200 μl/well) on ELISA plates (Nunc, Roskilde, Denmark) and incubated overnight at 4° C. The plates were blocked with PBS containing 5% (w/v) nonfat milk for 3 h at 37° C. and washed four times with PBS containing 0.05% Tween. Two-fold serially diluted mice sera were added into the wells and incubated for 1 h at 37° C. The plates were washed six times with PBS containing 0.05% Tween and incubated with HRP-conjugated goat anti-mouse IgG/IgG1/IgG2a for 1 h at 37° C. The color was developed using TMB solution (Sigma) and absorbance was measured using an ELISA reader at 450 nm. The antibody endpoint titer was defined as the serum dilution that produced an OD450 of 0.5 absorbance units in the ELISA assay.

ELISPOT assay. Mice were sacrificed 5 days after the third immunization. IFN-γ or IL17A producing splenocytes from vaccinated or naïve unvaccinated mice were analyzed using cytokine-specific enzyme-linked immunospot assay (ELISPOT) (BD PharMingen, United States). Briefly, plates were coated with capture antibodies (anti-IFN-γ or IL17A mAb) overnight at 4° C. and then blocked with a blocking solution (RPMI 1640 containing 10% fetal bovine serum and 1% L-glutamine-streptomycin-penicillin) for 1 h at 37° C. Splenocytes isolated from immunized mice were plated at a concentration of 1×10⁵ cells/well and stimulated with rSaEsxA (IMSA, 0.2 μg/well) or rSaEsxB (IMSB, 0.2 μg/well) at a final concentration of 10 μg/ml in triplicate and incubated for 20 h at 37° C. Ionomycin (1 μg/ml) (Sigma, United States) and phorbol myristate acetate (PMA, 50 ng/ml) (Sigma) were used as positive controls. Splenocytes from naïve mice stimulated with rSaEsxA (NMSA) or rSaEsxB (NMSB), splenocytes from unstimulated mice (immunized mice, UNS) and RPMI 1640 treated splenocytes were used as negative controls. After washing, biotinylated anti-IFN-γ or IL17A mAb was added for 1 h at 37° C., followed by streptavidin-HRP conjugate for 1 h at 37° C. The color was developed with TMB solution and the spots were counted using an immunospot analyzer.

Renal Abscess. S. aureus strain ATCC 25923 was plated onto a TSA plate with 5% horse blood and cultured for 24 h at 37° C. The bacteria were harvested using endotoxin-free PBS, washed twice, and suspended in PBS at a concentration of 5×10⁷ CFU/mL. On day 42 after the first vaccination, mice immunized with rSaEsxA (50 μg) or rSaEsxB (50 μg) were injected with 200 μl of the inoculums by i.p. at a total bacterial suspension concentration of 1×10⁷ CFU. Four days after bacterial challenge, infected mice were euthanized by compressed CO₂ inhalation. The kidneys were removed and homogenized in 1% Triton X-100. Aliquots were diluted and plated on blood agar for CFU counting. Kidney tissue samples for histological analysis were incubated in 10% formalin for 24 h at room temperature. Tissues were embedded in paraffin and thin sections were obtained using a microtome. Sections were stained with hematoxylin-eosin and examined under a microscope.

Lethal Challenge. On day 42 after the first vaccination, immunized mice were injected intravenously in the tail vein with 5×10⁷ CFU of S. aureus ATCC 25923, Newman (MSSA) or USA 300 (CA-MRSA) strains. Mice were monitored for mortality and clinical signs.

Passive immunization. Mouse polyclonal SaEsxA- or SaEsxB-specific antisera were generated and collected from the mice immunized with purified rSaEsxA or rSaEsxB. Female BALB/c mice (˜8 weeks old) were administered 100 μL of mouse normal sera or polyclonal SaEsxA- or SaEsxB-specific antisera (˜1:200000 antibody titers) by i.p. injection 4 h before S. aureus challenge and then 2 days after S. aureus challenge. Mice were monitored for mortality and clinical signs.

Statistical analysis. Student's t-test was used to analyze the statistical significance of ELISPOT assay results and Staphylococcal load measurements. Log-rank (Mantel-Cox) analysis was used to analyze the statistical significance of the data from the lethal challenge experiments. Analyses were performed using GraphPad Prism 5 (GraphPad Software, United States) and a p value<0.05 was considered statistically significant.

### EXAMPLE 2

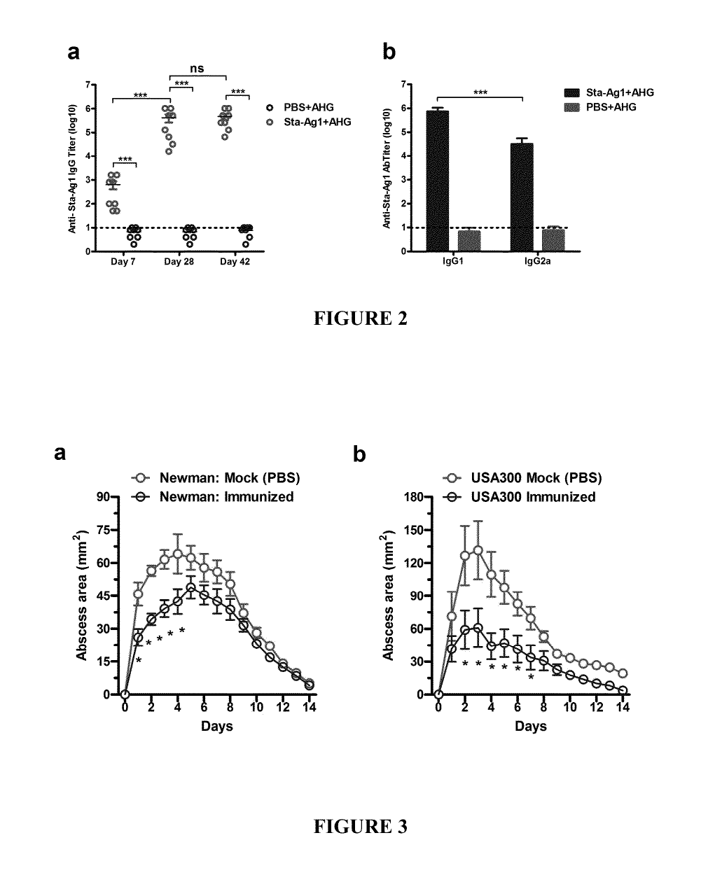

Targeting Sta-Ag1 (AdsA) for immunotherapeutic drug development. A fragment of Sta-Ag1 (36-430 aa that contained two 5′-nucleotidase motifs; DNA sequence from 106 to 1290 bp) was expressed and purified in E. coli, and the immunogenicity of the Sta-Ag1 protein was tested in a BALB/c mouse model. Mice vaccinated with Sta-Ag1 produced antibodies specific for the protein, as determined by ELISA. Half-maximal anti-Sta-Ag1 antibody titers were about 1:500,000 for the vaccinated mouse group, whereas anti-Sta-Ag1 was undetectable in the mock group (FIG. 2a); furthermore, immunization with Sta-Ag1 in the presence of aluminium hydroxide gel (AHG) also elicited both Th1 and Th2-associated rAdsA-specific IgG2a and IgG1 antibody responses (FIG. 2b).

Active immunization. Immunization with Sta-Ag1 moderates severity of USA300 skin infections. Recently, research has indicated that adenosine synthase A (AdsA, Sta-Ag1), a S. aureus cell wall-anchored enzyme, acts as an immune evasion factor. When both wild-type and AdsA mutant Staphylococci are mixed with fresh mouse or human blood, they are phagocytized by polymorphonuclear leukocytes (PMNs), particularly phagocytic neutrophils; however, wild-type Staphylococci survive within PMNs, but AdsA mutants are killed.

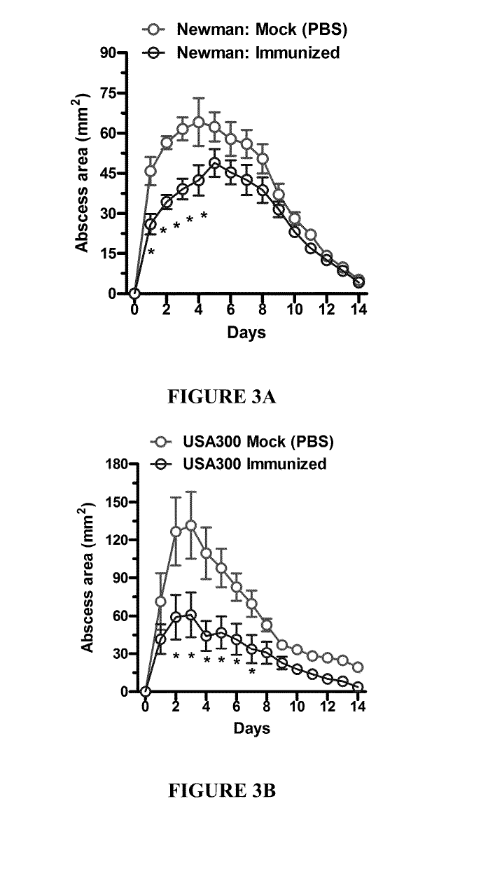

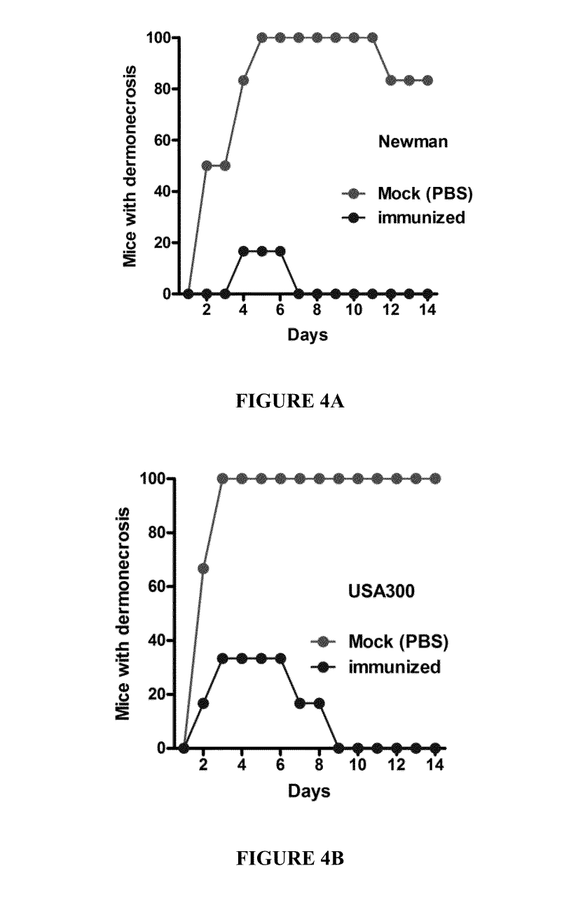

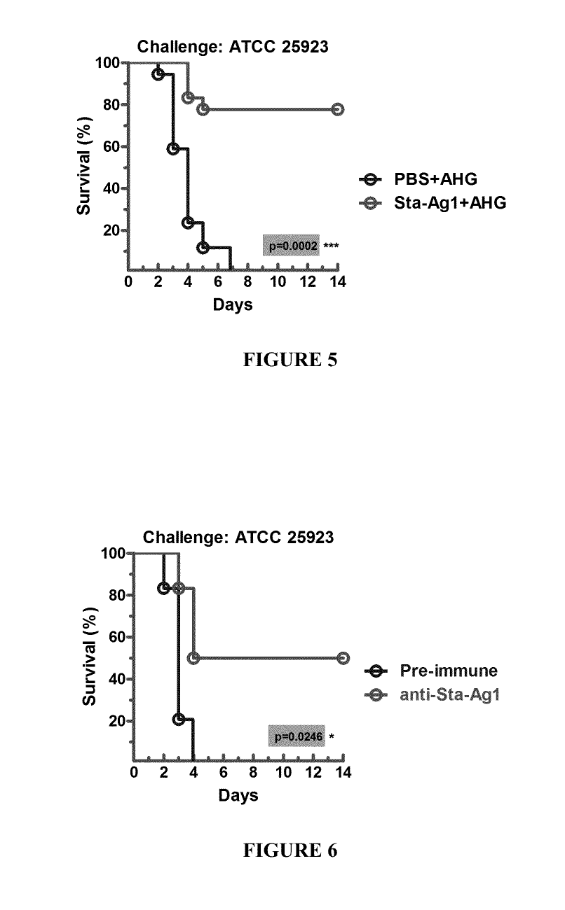

To determine whether active immunization with Sta-Ag1 protects mice from severe S. aureus lethal or skin infections, mice were vaccinated intramuscularly with Sta-Ag1+AHG 35 days before infection with USA 300 or Newman strains. The results showed that S. aureus abscess size was reduced significant in mice vaccinated with Sta-Ag1 (FIG. 3A and FIG. 3B). Also, there was little or no dermonecrosis in infected mice that had been vaccinated (FIGS. 4A, 4B, 4C), which demonstrates that active immunization with Sta-Ag1 moderates severity of S. aureus skin infections. Additionally, immunization of mice with Sta-Ag1 generated protective immunity against S. aureus lethal challenge in BalB/c mouse models (FIG. 5).

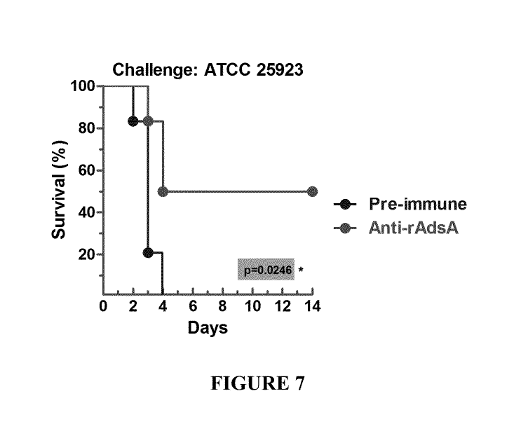

Passive immunization. Since active immunization with Sta-Ag1 moderates severity of USA300 skin infections and increases survival rate in a lethal infection model, it was next determined whether passive immunization with rabbit and mouse sera directed against Sta-Ag1 would moderate the severity of skin disease and reduce mortality in lethal challenge of a mouse model. The studies demonstrate that intraperitoneal injection of Sta-Ag1-specific rabbit anti-sera protects mice from lethal S. aureus infection (FIG. 6). The studies also demonstrate that intraperitoneal injection of Sta-Ag1-specific mouse anti-sera protects mice from lethal S. aureus infection (FIG. 7).

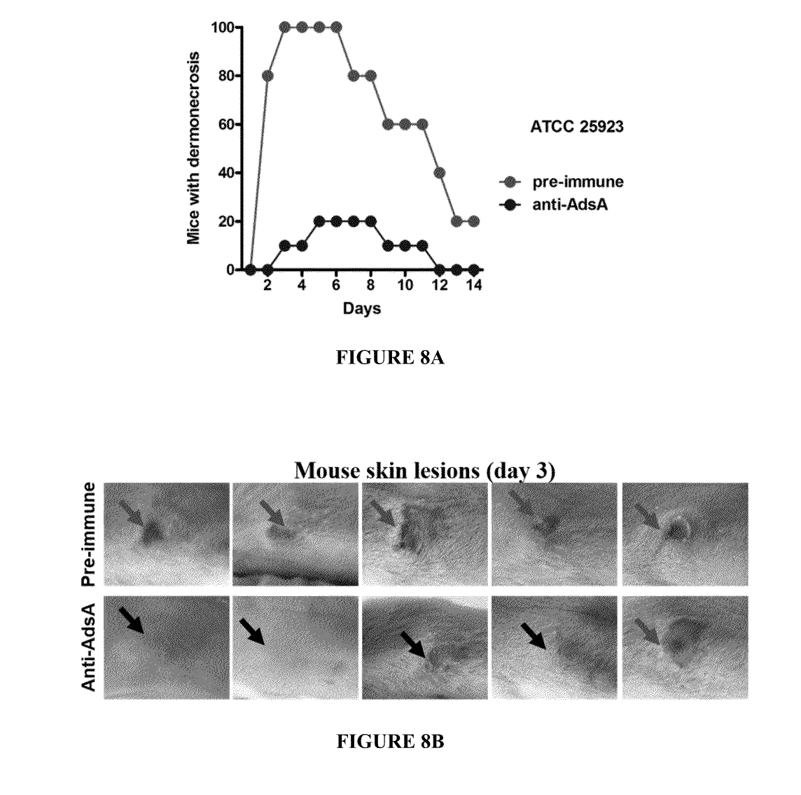

Skin lesions of mice infected with ATCC 25923 strains were significantly smaller after passive immunization with AdsA-specific mouse antisera, compared with lesions of mice that received pre-immune serum samples. Mice that received AdsA-specific mouse antisera either failed to develop dermonecrotic lesions after infection with ATCC 25923 strains or the area of dermonecrosis was limited (FIGS. 8A and 8B).

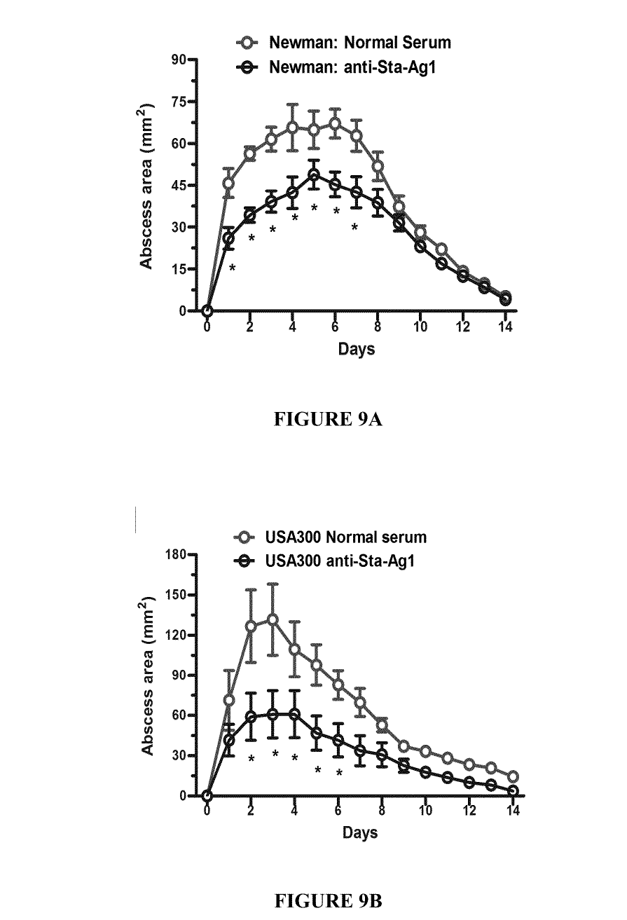

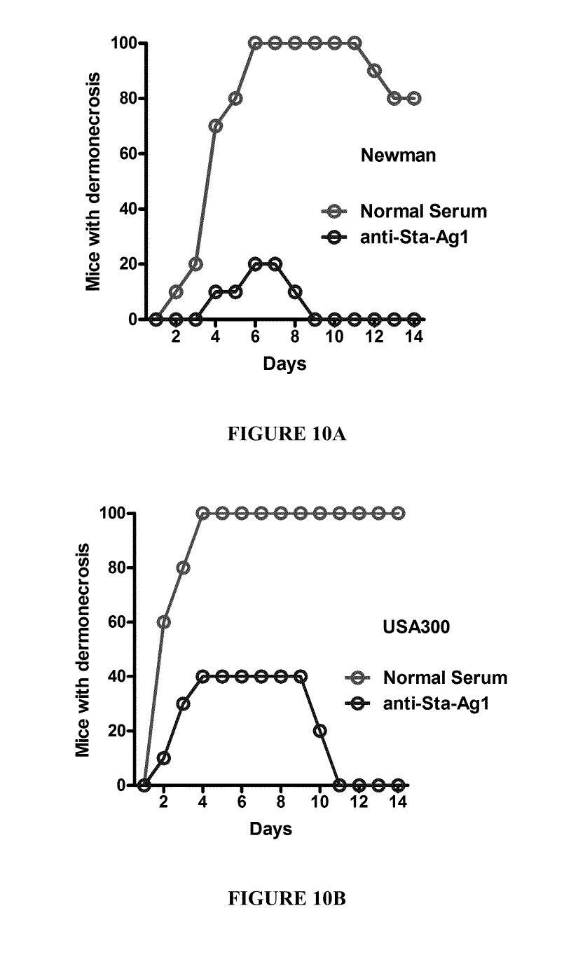

Skin lesions of mice infected with USA300 or Newman strains were significantly smaller after passive immunization with Sta-Ag1-specific rabbit antisera, compared with lesions of mice that received normal serum samples (FIGS. 9A and 9B). In addition, mice that received Sta-Ag1-specific rabbit antisera either failed to develop dermonecrotic lesions after infection with USA300 or Newman strains or the area of dermonecrosis was limited (FIGS. 10A and 10B). EXAMPLE 2-Targeting Sta-Ag2-3 for immunotherapeutic drug development. Recombinant Sta-Ag2 or 3 protein was expressed in E. coli BL21 and purified using a three-step chromatography strategy. Results indicated that the majority of the Sta-Ag2 and Sta-Ag3 were expressed in soluble form at a high yield (>99%). To evaluate the immunogenicity of Sta-Ag2 or Sta-Ag3, mice were vaccinated i.p. with three doses of Sta-Ag2 or Sta-Ag3 protein. Serum samples obtained 7 days following each immunization were evaluated by ELISA to assess the development of the antibody response. The Sta-Ag2 or Sta-Ag3 specific IgG, IgG1 and IgG2a antibody titers were evaluated by ELISA. The data shows that immunization with the Sta-Ag2 or Sta-Ag3 results in the generation of specific antibodies. Table 1 shows that IgG antibody titers were increased with raising doses of protein. Therefore, mice immunized with 50 ptg Sta-Ag2 (rSaEsxA) or Sta-Ag3 (rSaEsxB) (+FA or AHG) produced the highest titers on day 35.

TABLE 1
Immunization for different dose and forms of Sta-Ag2 or Sta-Ag3 against S. aureus infection

| | | | Titer^{b} | | | |
|---|---|---|---|---|---|---|
| | Dose | | Anti-Sta-Ag2 | Anti-Sta-Ag2 | Anti-Sta-Ag2 | Anti-Sta-Ag3 |
| Treatment | (μg) | Adjuvant^{a} | IgG | IgG1 | IgG2a | IgG |
| Sta-Ag2 | 50 | N | 2.0 × 10⁴ ± 3.0 × 10³ | 1.5 × 10⁴ ± 3.9 × 10³ | 8.0 × 10³ ± 1.5 × 10² | <20 |
| | 50 | AHG | 8.5 × 10⁴ ± 1.1 × 10⁴ | 5.9 × 10⁴ ± 1.2 × 10⁴ | 9.7 × 10⁴ ± 2.5 × 10² | <20 |
| | 50 | FA | 9.6 × 10⁴ ± 1.1 × 10⁴ | 6.4 × 10⁴ ± 1.1 × 10⁴ | 1.1 × 10⁴ ± 2.3 × 10² | <20 |
| | 10 | FA | 7.2 × 10⁴ ± 2.5 × 10⁴ | ND | ND | <20 |
| | 3 | FA | 5.4 × 10⁴ ± 3.0 × 10⁴ | ND | ND | <20 |
| Sta-Ag3 | 50 | N | <50 | ND | ND | 1.2 × 10³ = 3.1 × 10⁴ |
| | 50 | AHG | <50 | ND | ND | 1.2 × 10⁵ = 1.8 × 10⁵ |
| | 50 | FA | <50 | ND | ND | 2.6 × 10⁶ ± 1.9 × 10⁵ |
| | 10 | FA | <10 | ND | ND | 2.6 × 10⁶ ± 8.0 × 10⁴ |
| | 3 | FA | <10 | ND | ND | 1.9 × 10⁴ ± 6.4 × 10⁴ |
| Sta-Ag2 + 3 | 50 | AHG | 8.0 × 10⁴ ± 1.1 × 10⁴ | 6.9 × 10⁴ ± 1.0 × 10⁴ | 1.2 × 10⁴ ± 2.1 × 10⁴ | 1.3 × 10⁶ ± 1.9 × 10¹ |
| PBS mock | 0 | FA | <10 | <10 | <10 | <10 |
| | 0 | AHG | <10 | <10 | <10 | <10 |
| | 0 | N | <10 | <10 | <10 | <10 |

| | | | Titer^{b} | | | |
|---|---|---|---|---|---|---|
| | Dose | | Anti-Sta-Ag3 | Anti-Sta-Ag3 | | |
| Treatment | (μg) | Adjuvant^{a} | IgG1 | IgG2a | Survival^{c} | Significance^{d} |
| Sta-Ag2 | 50 | N | ND | ND | 66.70% | P < 0.05 |
| | 50 | AHG | ND | ND | 88.90% | P < 0.0001 |
| | 50 | FA | ND | ND | 83.30% | P < 0.0001 |
| | 10 | FA | ND | ND | 75.00% | P < 0.005 |
| | 3 | FA | ND | ND | 75.00% | P < 0.005 |
| Sta-Ag3 | 50 | N | 9.6 × 10⁴ ± 1.4 × 10² | 4.3 × 10³ ± 6.7 × 10⁴ | 50.00% | P < 0.05 |
| | 50 | AHG | 1.1 × 10⁴ ± 1.7 × 10² | 3.3 × 10⁵ ± 4.5 × 10⁴ | 77.78% | P < 0.0001 |
| | 50 | FA | 1.8 × 10⁴ ± 1.4 × 10⁵ | 3.8 × 10⁴ ± 4.4 × 10⁴ | 83.30% | P < 0.0001 |
| | 10 | FA | ND | ND | 62.50% | P < 0.003 |
| | 3 | FA | ND | ND | 50.00% | P < 0.05 |
| Sta-Ag2 + 3 | 50 | AHG | 1.0 × 10³ ± 1.7 × 10³ | 6.0 × 10⁴ ± 1.2 × 10⁴ | 83.30% | P < 0.0001 |
| PBS mock | 0 | FA | <10 | <10 | 22.20% | P = 0.7861 |
| | 0 | AHG | <10 | <10 | 16.70% | P = 0.7250 |
| | 0 | N | <10 | <10 | 11.10% | — |

^{a}FA: Freund's adjuvant.
AHG: Aluminium hydroxide gel;
N: No adjuvant
^{b}Antibody titers (titers ± SEM) were detected by ELISA with purified Sta-Ag2 or Sta-Ag3 (1 μg/ml); the antibody endpoint titer was defined as the serum dilution that produced an OD450 of 0.5 absorbance units in the ELISA assay.
^{c}Vaccinated BALB/c mice were challenged with S. aureus ATCC 2593 (5 × 10⁷ CFU) by intravenous injection and survival was monitored.
^{d}Log-rank (Mantel-Cox) test was used to compare between PBS mock (No adjuvant) and treatment groups.
ND: Not determined

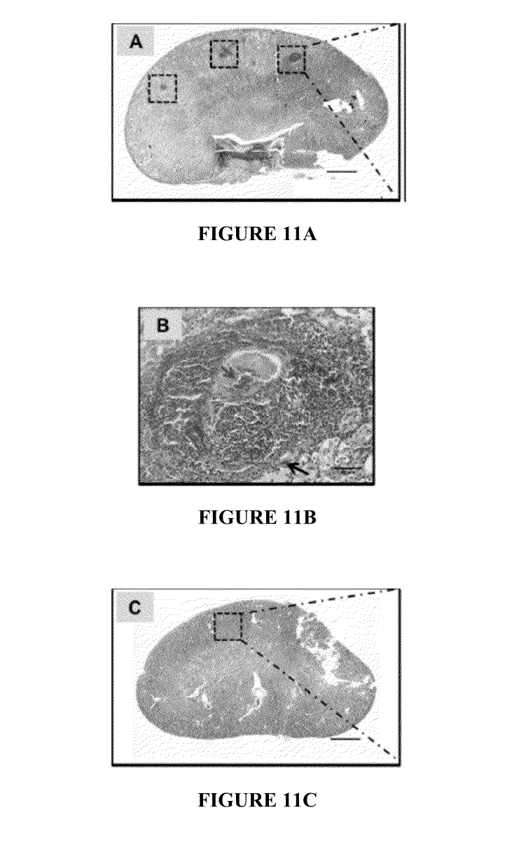

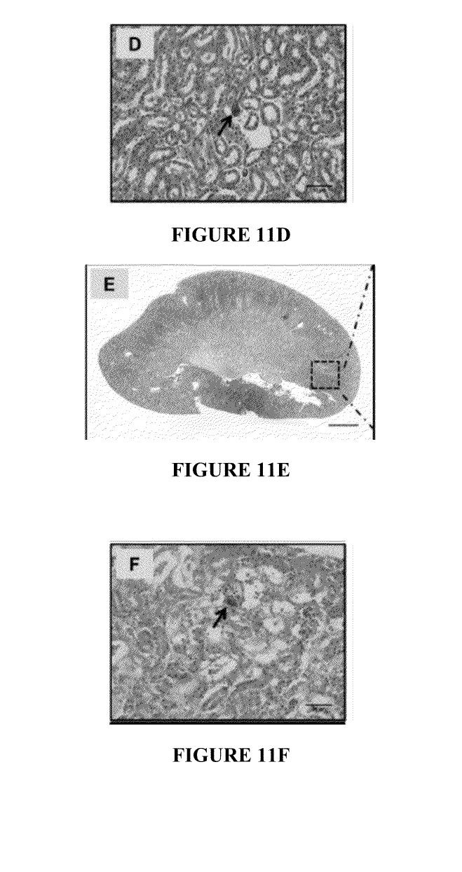

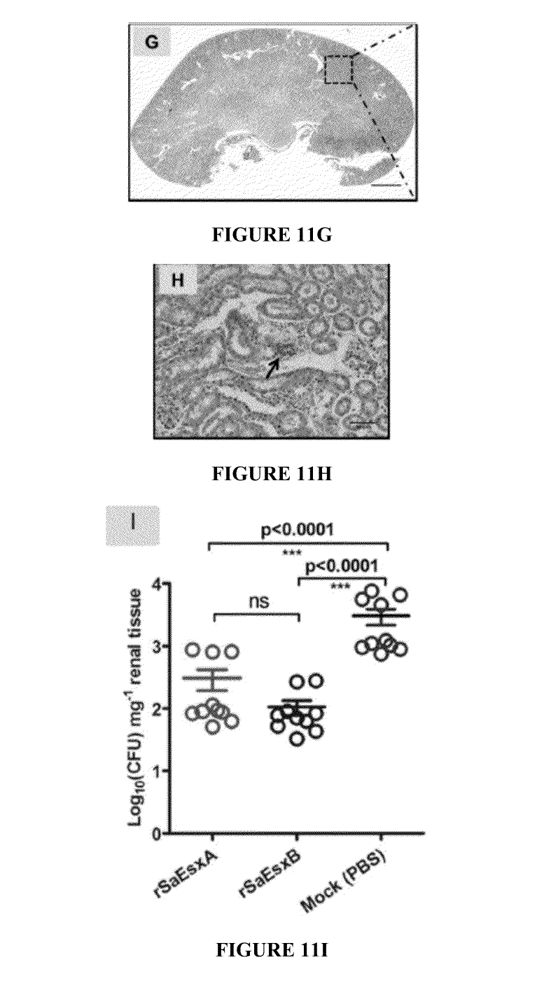

Renal Abscess. The potential protective effect of rSaEsxA and rSaEsxB in a mouse renal abscess model was evaluated. Mice were infected with 1×10⁷ CFU of S. aureus strain ATCC 25923. Four days after challenge, mice were sacrificed and their kidneys were collected. Renal tissue of animals treated with PBS displayed a staphylococcal load of 3.50 (+0.29) log 10 CFU mg⁻¹ of kidney tissues. Significant decreases in bacterial number were observed for animals treated with rSaEsxA [2.38 (+0.31) log 10 CFU mg⁻¹, p=0.0159] and rSaEsxB [1.97 (+0.05) log 10 CFU mg⁻¹, p=0.0079](FIG. 11I). Histological analysis of kidney tissues failed to detect staphylococcal abscesses in animals that had been immunized with the rSaEsxA+B or rSaEsxA and rSaEsxB alone (F 11C/FIG. 11D, FIG. 11E/FIG. 11F and FIG. 11G/FIG. 11H). Conversely, kidneys collected from control mice harbored abscesses with central concentrations of staphylococci that were surrounded by large numbers of necrotic immune cells (FIG. 11A/FIG. 11B).

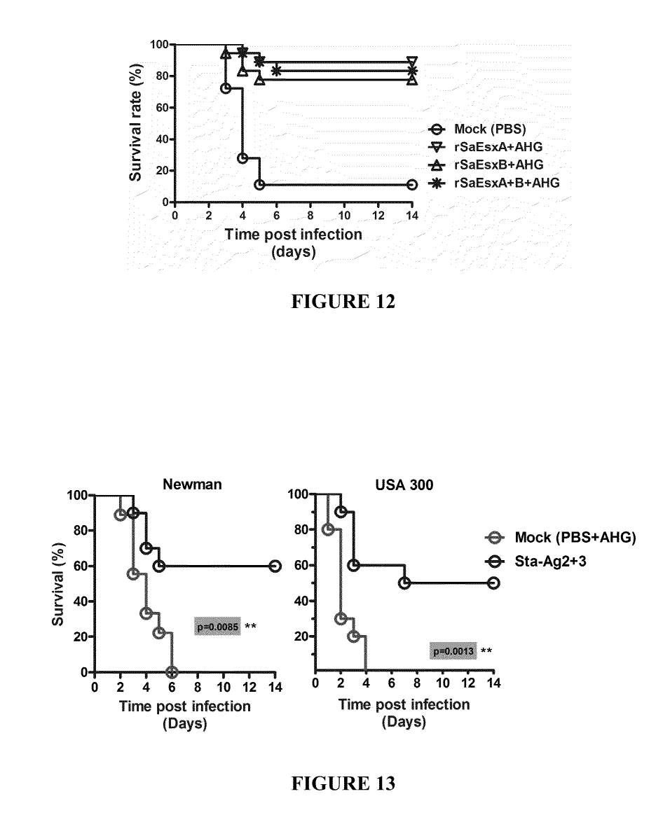

Lethal Challenge. The protective effect of the recombinant Sta-Ag2 or Sta-Ag3 proteins against lethal infections was investigated in mice immunized with purified rSaEsxA or rSaEsxB antigens, or a mixture of both. The immunized mice were challenged with 5×10′ CFU of S. aureus ATCC 25923 by intravenous injection through the tail vein. Animals were monitored for more than 14 days. Survival rates between groups were compared using the pair-wise, Log-rank (Mantel-Cox) test. The different survival rates of mice immunized with different treatments (protein+AHG or FA) and doses of Sta-Ag2 or Sta-Ag3 (3, 10 and 50 μg) against S. aureus ATCC 25923 are shown in Table 1. Survival rates increased with increasing doses of Sta-Ag2 or Sta-Ag3, with AHA or FA possibly playing non-specific roles against S. aureus infection. However, there were no significant differences between the adjuvant only groups (FA: 22.20%, P=0.7861; AHG: 16.70%, P=0.7250) and the PBS control group (11.10%). The results in FIG. 12 showed the vaccinated mice groups had significantly improved survival rates (p<0.0001). Specifically, mice vaccinated with Sta-Ag2 alone had the highest survival rate (16/18) compared to (14/18) for Sta-Ag3 alone and Sta-Ag2+3 vaccinated animals. In contrast, the majority of mice (16/18) in the control group died within 8 days after bacterial challenge. The survival rates between combined and individual antigens were not significantly different (p>0.05).

To test whether Sta-Ag2+3 could protect against a wide range of S. aureus clinical strains, two typical S. aureus strains, Newman and USA 300, were first tested. Newman is a methicillin-sensitive S. aureus strain and USA 300 is a community-associated methicillin-resistant S. aureus strain. Mice were treated with these strains in a similar manner to the above experiments and were monitored over 14 days. Compared with the control mice treated with PBS+AHG, mice vaccinated with combined Sta-Ag2+3 had significant protective immunity to Newman (60%; p=0.0085, Log-rank Mantel-Cox test) and USA 300 (50%; p=0.0013, Log-rank Mantel-Cox test) S. aureus strains (FIG. 13).

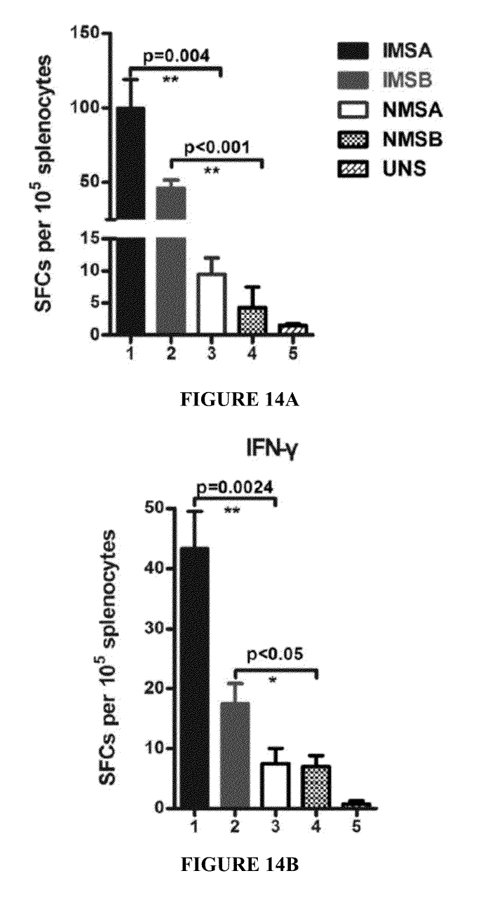

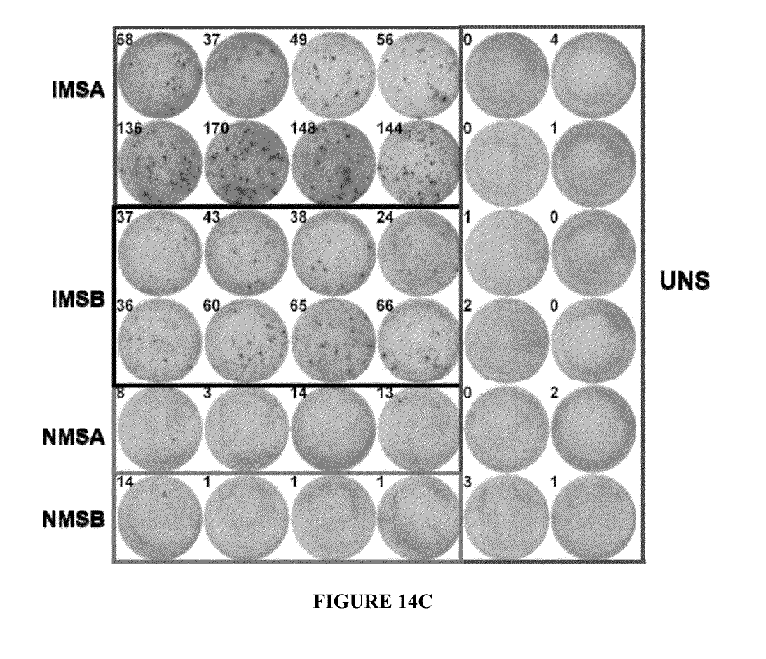

Sta-Ag2 or Sta-Ag3-specific IFN-γ⁺ and IL17A⁺ T cell responses. IFN-γ and IL17A play essential roles in the protective immunity against S. aureus infection. The release of IFN-γ and IL17A are indicative of Th1- and Th17-biased immune responses [33]. Mice were sacrificed 5 days after the third immunization and splenocyte production of IFN-γ and IL17A cytokines was measured by ELISPOT. Splenocytes from mice immunized with Sta-Ag2 or Sta-Ag3 had more IFN-γ (FIG. 14A) and IL17A (FIG. 14B) producing cells compared to the control group. The numbers of IFN-γ-producing splenocytes in these immunized mice were also significantly greater than in NMSA (naïve mice stimulated with Sta-Ag2) and NMSB (naïve mice stimulated with Sta-Ag3). Furthermore, immunization with Sta-Ag2 or Sta-Ag3 induced robust specific Th17 responses.

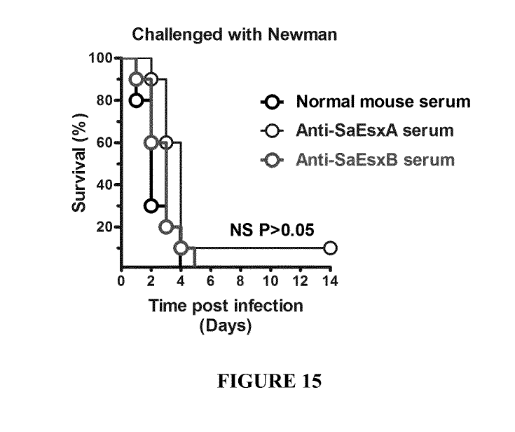

Finally, mice treated with SaEsxA- or SaEsxB-specific antisera did not exhibit any significant protective effects against S. aureus challenge (p>0.05, Log-rank Mantel-Cox test) (FIG. 15). Treatment with SaEsxA- or SaEsxB-specific antisera alone could not provide effective immunity.

Taken together, these data indicate that SaEsxA or SaEsxB proteins promote the induction of Th1- and Th17-biased immune responses, but SaEsxA- or SaEsxB-specific antisera alone could not provide effective immunity.

### EXAMPLE 3

Targeting Sta-Ag4-11 for drug development. Phenol-soluble modulins (PSMs) have recently emerged as a novel toxin family defining the virulence potential of highly aggressive S. aureus isolates. PSMs have multiple roles in staphylococcal pathogenesis, causing lysis of red and white blood cells, stimulating inflammatory responses and contributing to biofilm development and the dissemination of biofilm-associated infections [34, 35]. Moreover, the pronounced capacity of PSMs to kill human neutrophils after phagocytosis might explain failures in the development of anti-staphylococcal vaccines. In S. aureus, however, not all PSMs are cytolytic. The four PSMα peptides of S. aureus, PSMα1 (Sta-Ag5), PSMα2 (Sta-Ag6), PSMα3 (Sta-Ag7), and PSMα4 (Sta-Ag8), have a pronounced ability to lyse human leukocytes and erythrocytes, and PSMα3 has by far the strongest activity [35]. All PSMs are secreted without a signal peptide, carrying an amino terminal N-formyl methionine. The lack of a signal peptide indicates that PSM secretion takes place by a dedicated mechanism, which was recently identified to be a four-component ABC transporter, named PmtA (Sta-Ag9), PmtB (Sta-Ag10), PmtC (Sta-Ag111) and PmtD (Sta-Ag12). This transporter plays an essential part in S. aureus physiology because in its absence, PSM peptides accumulate in the cytosol, leading to cell death.

Targeting PSMs for vaccine development. Experiments led to the identification of S. aureus PSMs that kill human neutrophils and have a major impact on the ability of the recently emerged community-associated methicillin-resistant S. aureus (CA-MRSA) strains to cause disease [34, 35]. PSMα1 (Sta-Ag5), PSMα2 (Sta-Ag6), PSMα3 (Sta-Ag7), and PSMα4 (Sta-Ag8) can be used as potential antigens in active vaccination approaches. In addition, Pmt [PmtA (Sta-Ag9), PmtB (Sta-Ag10), PmtC (Sta-Ag11) and PmtD (Sta-Ag12)] can also be potential targets for active vaccination, given surface location and essential role in growth and pathogenesis. Therefore, based on PSMs associated antigens, they can be utilized to develop a vaccine which elicits a potential antibody to prevent skin infection.

Targeting PSMs for immunotherapeutic antibodies development. MAb-dependent facilitation of opsonophagocytosis might not lead to enhanced killing of S. aureus, however, mAbs can eliminate PSM toxicity by sequestration. In addition, drugs blocking the Pmt [PmtA (Sta-Ag9), PmtB (Sta-Ag10), PmtC (Sta-Ag111) and PmtD (Sta-Ag12)]transport function should work on all PSM-producing species, as the Pmt system is well conserved.

### EXAMPLE 4

Novel combined vaccines targeting Sta-Ag1, Sta-Ag2, Sta-Ag3, and Sta-Ag4-12 Th17 cell targets (Sta-Ag2, 3) and B cell targets (Sta-Ag1, 4-12) are combined to develop a novel vaccine, thereby acting synergistically to effectively prevent S. aureus hematic spread, pneumonia, skin infection and mastitis in dairy cows. Despite eliciting high levels of anti-SaEsxA IgG and anti-SaEsxB IgG after vaccination with the purified SaEsxA and SaEsxB proteins, these antibodies could not prevent S. aureus infection in a murine model. Studies showed that healthy individuals naturally have high levels of antibody titers to S. aureus, but those with defects in B cell immunity are not particularly prone to S. aureus infections (36, 7). The lack of humoral immunity protection against S. aureus must be compensated for by other immune mechanisms.

To study if mice immunized with rSaEsxA or rSaEsxB could prevent abscess formation, a murine model of staphylococcal load and abscess formation was chosen. The results indicate that SaEsxA or SaEsxA proteins could induce protective immunity against S. aureus renal abscess formation in this murine model. Furthermore, these results suggest that mice immunized with the rSaEsxA+B, rSaEsxA or rSaEsxB had significantly increased protection against lethal challenge by S. aureus ATCC 25923 and show that rSaEsxA and rSaEsxB proteins not only specifically trigger high levels of IL-17A but also high levels of IFN-γ. These data indicate that SaEsxA and SaEsxB promote the induction of Th1- and Th17-biased immune responses. The results also showed that mice immunized with rSaEsxA+B were protected against two typical S. aureus clinical strains, Newman (MSSA) and USA 300 (CA-MRSA).

At least 13 secreted proteins and 24 surface adhesion proteins from S. aureus have been implicated in the bacterial immune evasion (38). The secretion of SaEsxA and SaEsxB represents an important bacterial virulence strategy, which leads to bacterial replication and abscess formation (32). Therefore, instead of a vaccine targeting a single-antigen, multivalent antigens are a better alternative for inducing both B and T cell immune responses to achieve protection against S. aureus.

### EXAMPLE 5

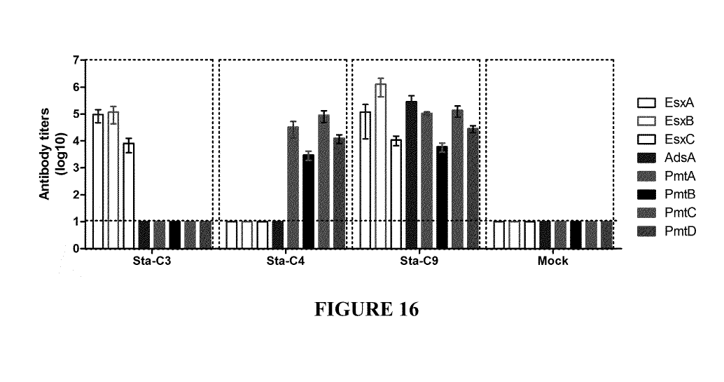

Mice were vaccinated with combinations of Sta-Ag1, Sta-Ag2, and Sta-Ag3 (Sta-C3), Sta-Ag9, Sta-Ag10, Sta-Ag11 and Sta-Ag12 (Sta-C4), or Sta-Ag1, Sta-Ag2, Sta-Ag3, Sta-Ag4, Sta-Ag9, Sta-Ag10, Sta-Ag11 and Sta-Ag12 (Sta-C9). Determination of antibody titers by ELISA revealed antibodies specific for Sta-Ag2, Sta-Ag3, and Sta-Ag4 in Sta-C3-treated animals, antibodies specific for Sta-Ag9, Sta-Ag10, Sta-Ag11 and Sta-Ag12 in Sta-C4-treated animals, and antibodies specific for Sta-Ag1, Sta-Ag2, Sta-Ag3, Sta-Ag4, Sta-Ag9, Sta-Ag10, Sta-Ag11 and Sta-Ag12 in Sta-C9-treated animals, respectively (FIG. 16). No antibodies were detected in mock treated animals.

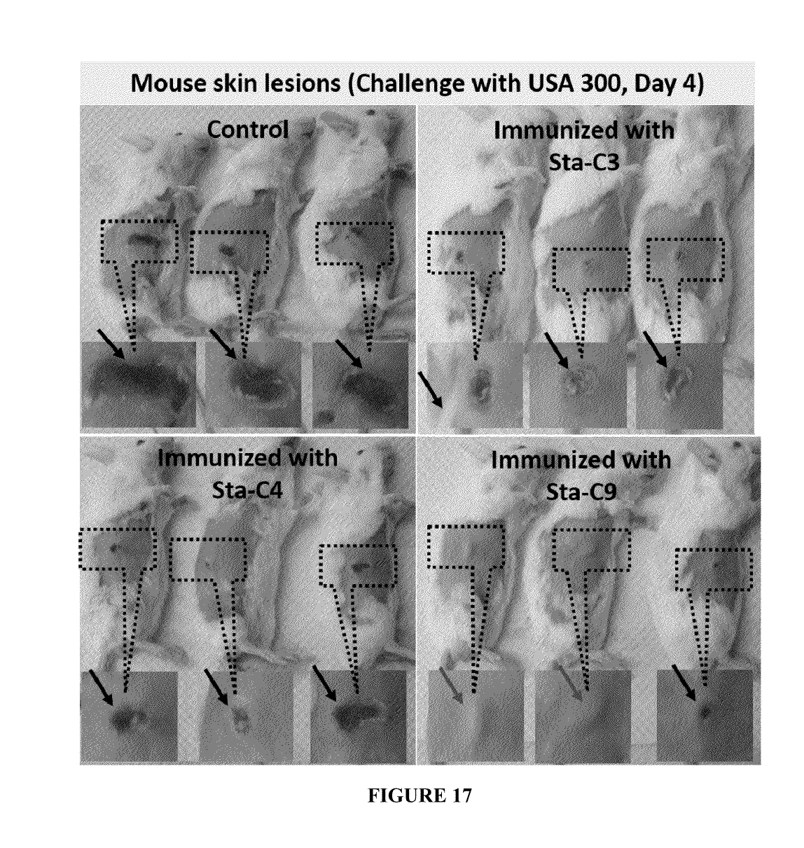

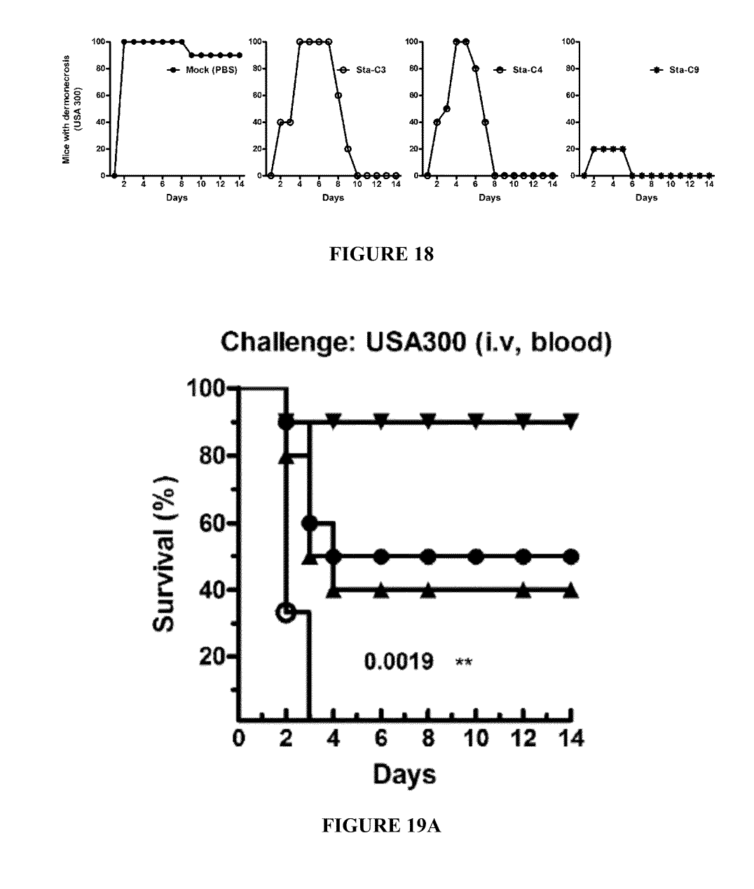

Active immunization. To determine whether active immunization with combinations of Sta-Ag2, Sta-Ag3, and Sta-Ag4 (Sta-C3), Sta-Ag9, Sta-Ag10, Sta-Ag11 and Sta-Ag12 (Sta-C4), or Sta-Ag1, Sta-Ag2, Sta-Ag3, Sta-Ag4, Sta-Ag9, Sta-Ag10, Sta-Ag1 and Sta-Ag12 (Sta-C9) protected mice from severe S. aureus skin infections, mice were vaccinated intramuscularly with Sta-C3, Sta-C4, or Sta-C9 before infection with the USA 300 strain. The results showed that S. aureus abscess size was reduced significant in mice vaccinated with Sta-C3, Sta-C4, and Sta-C9 and vaccinated mice showed substantially reduced dermonecrosis (FIG. 17). Remarkably, Sta-C9 prevented skin lesions in 80% of the mice (FIGS. 17 and 18).

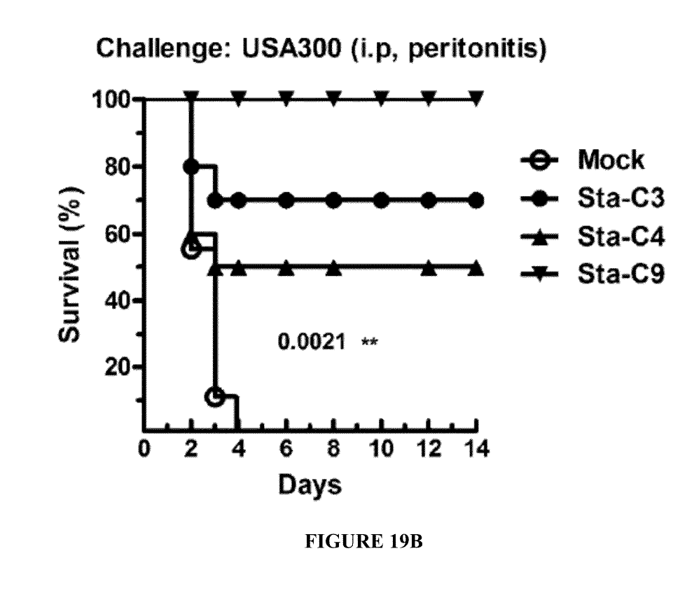

Lethal Challenge. The protective effect of Sta-C3, Sta-C4, and Sta-C9 against lethal infections was investigated in mice immunized with Sta-C3, Sta-C4, or Sta-C9. The immunized mice were challenged with USA300 either injected i.v. through the tail vein or i.p. Animals were monitored for more than 14 days. Survival rates between groups were compared using the pair-wise, Log-rank (Mantel-Cox) test. The results showed that vaccinated mice groups had significantly improved survival rates after i.v. challenge (p<0.0019) and i.p. challenge (p<0.0021). Specifically, mice vaccinated with Sta-C9 had the highest survival rate (90% after i.v. challenge and 100% after i.p. challenge) compared to Sta-C3 and Sta-C4. In contrast, the majority of mice in the control group died within 4 days after bacterial challenge. These results demonstrated that Sta-C3, Sta-C4, and Sta-C9 generated protective immunity against S. aureus lethal challenge in a mouse model (FIGS. 19A and 19B).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Kluytmans, J., van Belkum, A. & Verbrugh, H. Nasal carriage of Staphylococcus aureus: epidemiology, underlying mechanisms, and associated risks. Clinical microbiology reviews 10, 505-520 (1997).
2. Perl, T. M., et al. Intranasal mupirocin to prevent postoperative Staphylococcus aureus infections. The New England journal of medicine 346, 1871-1877 (2002).
3. Gould, I. M. The clinical significance of methicillin-resistant Staphylococcus aureus. The Journal of hospital infection 61, 277-282 (2005).
4. Abrahamian, F. M. & Moran, G. J. Methicillin-resistant Staphylococcus aureus infections. The New England journal of medicine 357, 2090; author reply 2090 (2007).
5. Schaffer, A. C. & Lee, J. C. Vaccination and passive immunisation against Staphylococcus aureus. International journal of antimicrobial agents 32 Suppl 1, S71-78 (2008).
6. Stranger-Jones, Y. K., Bae, T. & Schneewind, O. Vaccine assembly from surface proteins of Staphylococcus aureus. Proceedings of the National Academy of Sciences of the United States of America 103, 16942-16947 (2006).
7. Bubeck Wardenburg, J. & Schneewind, O. Vaccine protection against Staphylococcus aureus pneumonia. The Journal of experimental medicine 205, 287-294 (2008).
8. Yin, R. L., et al. Construction and immunogenicity of a DNA vaccine containing clumping factor A of Staphylococcus aureus and bovine IL18. Veterinary immunology and immunopathology 132, 270-274 (2009).
9. Jonsson, I. M., et al. On the role of Staphylococcus aureus sortase and sortase-catalyzed surface protein anchoring in murine septic arthritis. The Journal of infectious diseases 185, 1417-1424 (2002).
10. Inskeep, T. K., et al. Oral vaccine formulations stimulate mucosal and systemic antibody responses against staphylococcal enterotoxin B in a piglet model. Clinical and vaccine immunology: CVI 17, 1163-1169 (2010).
11. Kim, H. K., Cheng, A. G., Kim, H. Y., Missiakas, D. M. & Schneewind, O. Nontoxigenic protein A vaccine for methicillin-resistant Staphylococcus aureus infections in mice. The Journal of experimental medicine 207, 1863-1870 (2010).
12. Arrecubieta, C., et al. Vaccination with clumping factor A and fibronectin binding protein A to prevent Staphylococcus aureus infection of an aortic patch in mice. The Journal of infectious diseases 198, 571-575 (2008).
13. Kim, H. K., et al. IsdA and IsdB antibodies protect mice against Staphylococcus aureus abscess formation and lethal challenge. Vaccine 28, 6382-6392 (2010).
14. Roth, D. M., Senna, J. P. & Machado, D. C. Evaluation of the humoral immune response in BALB/c mice immunized with a naked DNA vaccine anti-methicillin-resistant Staphylococcus aureus. Genetics and molecular research: GMR 5, 503-512 (2006).
15. Gaudreau, M. C., Lacasse, P. & Talbot, B. G. Protective immune responses to a multi-gene DNA vaccine against Staphylococcus aureus. Vaccine 25, 814-824 (2007).
16. Staphylococcus aureus vaccine conjugate-Nabi: Nabi-StaphVAX, StaphVAX. Drugs in R&D 4, 383-385 (2003).
17. Jones, T. StaphVAX (Nabi). Curr Opin Investig Drugs 3, 48-50 (2002).
18. Fattom, A., et al. Safety and immunogenicity of a booster dose of Staphylococcus aureus types 5 and 8 capsular polysaccharide conjugate vaccine (StaphVAX) in hemodialysis patients. Vaccine 23, 656-663 (2004).
19. Shinefield, H., et al. Use of a Staphylococcus aureus conjugate vaccine in patients receiving hemodialysis. The New England journal of medicine 346, 491-496 (2002).
20. Menzies, B. E. & Kernodle, D. S. Site-directed mutagenesis of the alpha-toxin gene of Staphylococcus aureus: role of histidines in toxin activity in vitro and in a murine model. Infection and immunity 62, 1843-1847 (1994).
21. Spellberg, B., et al. The antifungal vaccine derived from the recombinant N terminus of Als3p protects mice against the bacterium Staphylococcus aureus. Infection and immunity 76, 4574-4580 (2008).
22. Brown, E. L., et al. The Panton-Valentine leukocidin vaccine protects mice against lung and skin infections caused by Staphylococcus aureus USA300. Clinical microbiology and infection: the official publication of the European Society of Clinical Microbiology and Infectious Diseases 15, 156-164 (2009).
23. Kuklin, N. A., et al. A novel Staphylococcus aureus vaccine: iron surface determinant B induces rapid antibody responses in rhesus macaques and specific increased survival in a murine S. aureus sepsis model. Infection and immunity 74, 2215-2223 (2006).
24. DeJonge, M., et al. Clinical trial of safety and efficacy of INH-A21 for the prevention of nosocomial staphylococcal bloodstream infection in premature infants. The Journal of pediatrics 151, 260-265, 265 e261 (2007).
25. Patti, J. M. A humanized monoclonal antibody targeting Staphylococcus aureus. Vaccine 22 Suppl 1, S39-43 (2004).
26. Benjamin, D. K., et al. A blinded, randomized, multicenter study of an intravenous Staphylococcus aureus immune globulin. Journal of perinatology: official journal of the California Perinatal Association 26, 290-295 (2006).
27. Rupp, M. E., et al. Phase II, randomized, multicenter, double-blind, placebo-controlled trial of a polyclonal anti-Staphylococcus aureus capsular polysaccharide immune globulin in treatment of Staphylococcus aureus bacteremia. Antimicrobial agents and chemotherapy 51, 4249-4254 (2007).
28. Proctor, R. A. Challenges for a universal Staphylococcus aureus vaccine. Clinical infectious diseases: an official publication of the Infectious Diseases Society of America 54, 1179-1186 (2012).
29. Thammavongsa, V., Kern, J. W., Missiakas, D. M. & Schneewind, O. Staphylococcus aureus synthesizes adenosine to escape host immune responses. The Journal of experimental medicine 206, 2417-2427 (2009).
30. Thammavongsa, V., Schneewind, O. & Missiakas, D. M. Enzymatic properties of Staphylococcus aureus adenosine synthase (AdsA). BMC biochemistry 12, 56 (2011).
31. Kim, H. K., Thammavongsa, V., Schneewind, O. & Missiakas, D. Recurrent infections and immune evasion strategies of Staphylococcus aureus. Current opinion in microbiology 15, 92-99 (2012).
32. Burts, M. L., Williams, W. A., DeBord, K. & Missiakas, D. M. EsxA and EsxB are secreted by an ESAT-6-like system that is required for the pathogenesis of Staphylococcus aureus infections. Proceedings of the National Academy of Sciences of the United States of America 102, 1169-1174 (2005).
33. Lin, L., et al. Th1-Th17 cells mediate protective adaptive immunity against Staphylococcus aureus and Candida albicans infection in mice. PLoS pathogens 5, e1000703 (2009).
34. Chatterjee, S. S., et al. Essential Staphylococcus aureus toxin export system. Nat Med 19, 364-367 (2013).
35. Peschel, A. & Otto, M. Phenol-soluble modulins and staphylococcal infection. Nature reviews. Microbiology 11, 667-673 (2013).
36. Holland S M. 2010. Chronic granulomatous disease. Clin Rev Allergy Immunol 38:3-10.http://dx.doi.org//10.1007/s12016-009-8136-z
37. Roos D, Kuhns D B, Maddalena A, Roesler J, Lopez J A, Ariga T, Avcin T, de Boer M, Bustamante J, Condino-Neto A, Di Matteo G, He J, Hill H R, Holland S M, Kannengiesser C, Koker M Y, Kondratenko I, van Leeuwen K, Malech H L, Marodi L, Nunoi H, Stasia M J, Ventura A M, Witwer C T, Wolach B, Gallin J I. 2010. Hematologically important mutations: X-linked chronic granulomatous disease (third update). Blood Cells Mol Dis 45:246-265.http://dx.doi.org//10.1016/j_bcmd.2010.07.012
38. McCarthy A J, Lindsay J A. 2010. Genetic variation in Staphylococcus aureus surface and immune evasion genes is lineage associated: implications for vaccine design and host-pathogen interactions. BMC Microbiol 10:173.http://dx.doi.org//10.1186/1471-2180-10-173
39. Sorensen A L, Nagai S, Houen G, Andersen P, Andersen A B. 1995. Purification and characterization of a low-molecular-mass T-cell antigen secreted by Mycobacterium tuberculosis. Infect Immun 63:1710-1717
40. Chatterjee S, Dwivedi V P, Singh Y, Siddiqui I, Sharma P, Van Kaer L, Chattopadhyay D, Das G. 2011. Early secreted antigen ESAT-6 of Mycobacterium tuberculosis promotes protective T helper 17 cell responses in a toll-like receptor-2-dependent manner. PLoS Pathog 7:e1002378.http://dx.doi_org//10.1371/journal.ppat. 1002378
41. Pearce E J, Caspar P, Grzych J M, Lewis F A, Sher A. 1991. Downregulation of Th1 cytokine production accompanies induction of Th2 responses by a parasitic helminth, Schistosoma mansoni. J Exp Med 173:159-166.http://dx.doi.org//10.1084/jem.173.1.159
42. Misstear K, McNeela E A, Murphy A G, Geoghegan J A, O'Keeffe K M, Fox J, Chan K, Heuking S, Collin N, Foster T J, McLoughlin R M, Lavelle E C. 2014. Targeted nasal vaccination provides antibody-independent protection against Staphylococcus aureus. J Infect Dis 209: 1479-1484.http://dx.doi.org//10.1093/infdis/jit636
43. Miller L S, Cho J S. 2011. Immunity against Staphylococcus aureus cutaneous infections. Nat Rev Immunol 11:505-518.http://dx.doi.org//10.1038/nri3010
44. Minegishi Y, Karasuyama H. 2009. Defects in Jak-STAT-mediated cytokine signals cause hyper-IgE syndrome: lessons from a primary immunodeficiency. Int Immunol 21:105-112.http://dx.doi.org/10.1093/intimm/dxn134
45. Kumar P, Chen K, Kolls J K. 2013. Th17 cell based vaccines in mucosal immunity. Curr Opin Immunol 25:373-380.http://dx.doi.org//10.1016/j_coi.2013.03011
46. Ulrichs T, Munk M E, Mollenkopf H, Behr-Perst S, Colangeli R, Gennaro M L, Kaufmann S H. 1998. Differential T cell responses to Mycobacterium tuberculosis ESAT6 in tuberculosis patients and healthy donors. Eur J Immunol 28:3949-3958.http://dx.doi.org//10.1002/(SICI)1521-4141(199812)28:12&#60:3949::AID-IMMU3949&#62.3.0.CO.2-4

What is claimed is:

1. A vaccine formulation, comprising:
a Staphylococcus aureus polypeptide selected from Sta-Ag1, Sta-Ag4, Sta-Ag5, Sta-Ag6, Sta-Ag7, Sta-Ag8, Sta-Ag9, Sta-Ag10, Sta-Ag11, and Sta-Ag12; any combination thereof; a combination of Sta-Ag2 with any of Sta-Ag1, Sta-Ag4, Sta-Ag5, Sta-Ag6, Sta-Ag7, Sta-Ag8, Sta-Ag9, Sta-Ag10, Sta-Ag11, and Sta-Ag12; or a combination of Sta-Ag3 with any of Sta-Ag1, Sta-Ag4, Sta-Ag5, Sta-Ag6, Sta-Ag7, Sta-Ag8, Sta-Ag9, Sta-Ag10, Sta-Ag11, and Sta-Ag12.

2. The vaccine formulation of claim 1, wherein the combination of two or more Staphylococcus aureus polypeptides achieves protection against hematic spread, pneumonia, or skin infection.

3. The vaccine formulation of claim 1 comprising Sta-Ag2, Sta-Ag3, and Sta-Ag4.

4. The vaccine formulation of claim 1 comprising Sta-Ag9, Sta-Ag10, Sta-Ag11, and Sta-Ag12.

5. The vaccine formulation of claim 1 comprising Sta-Ag1, Sta-Ag2, Sta-Ag-3, Sta-Ag4, Sta-Ag9, Sta-Ag100, Sta-Ag11, and Sta-Ag12.

6. The vaccine formulation of claim 1, further comprising at least one adjuvant.

7. The vaccine formulation of claim 6, wherein the adjuvant is selected from a Th1 adjuvant, a Th2 adjuvant, a Th17 adjuvant, and any combination thereof.

8. The vaccine formulation of claim 7, further comprising an aluminum hydroxide adjuvant.

9. The vaccine formulation of claim 8, further comprising a histidine buffer.

10. The vaccine formulation of claim 1, wherein the Sta-Ag1 Staphylococcus aureus polypeptide comprises the amino acid sequence of SEQ ID NO: 1.

11. The vaccine formulation of claim 1, wherein the Sta-Ag2 Staphylococcus aureus polypeptide comprises the amino acid sequence of SEQ ID NO:2.

12. The vaccine formulation of claim 1, wherein the Sta-Ag3 Staphylococcus aureus polypeptide comprises the amino acid sequence of SEQ ID NO:3.

13. The vaccine formulation of claim 1, wherein the Sta-Ag4 Staphylococcus aureus polypeptide comprises the amino acid sequence of SEQ ID NO:4.

14. The vaccine formulation of claim 1, wherein the Sta-Ag5 Staphylococcus aureus polypeptide comprises the amino acid sequence of SEQ ID NO:5.

15. The vaccine formulation of claim 1, wherein the Sta-Ag6 Staphylococcus aureus polypeptide comprises the amino acid sequence of SEQ ID NO:6.

16. The vaccine formulation of claim 1, wherein the Sta-Ag7 Staphylococcus aureus polypeptide comprises the amino acid sequence of SEQ ID NO:7.

17. The vaccine formulation of claim 1, wherein the Sta-Ag8 Staphylococcus aureus polypeptide comprises the amino acid sequence of SEQ ID NO:8.

18. The vaccine formulation of claim 1, wherein the Sta-Ag9 Staphylococcus aureus polypeptide comprises the amino acid sequence of SEQ ID NO:9.

19. The vaccine formulation of claim 1, wherein the Sta-Ag10 Staphylococcus aureus polypeptide comprises the amino acid sequence of SEQ ID NO: 10.

20. The vaccine formulation of claim 1, wherein the Sta-Ag11 Staphylococcus aureus polypeptide comprises the amino acid sequence of SEQ ID NO: 11.

21. The vaccine formulation of claim 1, wherein the Sta-Ag12 Staphylococcus aureus polypeptide comprises the amino acid sequence of SEQ ID NO: 12.

22. An isolated aptamer or antibody that binds with specificity to a polypeptide selected from a Sta-Ag1 Staphylococcus aureus polypeptide, a Sta-Ag2 Staphylococcus aureus polypeptide, a Sta-Ag3 Staphylococcus aureus polypeptide, a Sta-Ag4 Staphylococcus aureus polypeptide, a Sta-Ag5 Staphylococcus aureus polypeptide, a Sta-Ag6 Staphylococcus aureus polypeptide, a Sta-Ag7 Staphylococcus aureus polypeptide, a Sta-Ag8 Staphylococcus aureus polypeptide, a Sta-Ag9 Staphylococcus aureus polypeptide, a Sta-Ag10 Staphylococcus aureus polypeptide, a Sta-Ag11 Staphylococcus aureus polypeptide, and a Sta-Ag12 Staphylococcus aureus polypeptide.

23. A method for inhibiting Staphylococcus aureus infection in a subject, comprising: administering to the subject an effective amount of a vaccine formulation of claim 1.

24. A method for inhibiting Staphylococcus aureus infection in a subject, comprising: administering to the subject an effective amount of an antibody according to claim 22.


SEQUENCE LISTING (12 sequences)

SEQ ID NO: 1 - protein, 772 aa, Staphylococcus aureus
MKALLLKTSVWLVLLFSVMGLWQVSNAAEQHTPMKAHAVTTIDKATTDKQQVPPTKEAAHHSGKEAATNVSASAQGTADDTNSKVTSNAPSNKPSTVVSTKVNETRDVDTQQASTQKPTHTATFKLSNAKTASLSPRMFAANAPQTTTHKILHTNDIHGRLAEEKGRVIGMAKLKTVKEQEKPDLMLDAGDAFQGLPLSNQSKGEEMAKAMNAVGYDAMAVGNHEFDFGYDQLKKLEGMLDFPMLSTNVYKDGKRAFKPSTIVTKNGIRYGIIGVTTPETKTKTRPEGIKGVEFRDPLQSVTAEMMRIYKDVDTFVVISHLGIDPSTQETWRGDYLVKQLSQNPQLKKRITVIDGHSHTVLQNGQIYNNDALAQTGTALANIGKITFNYRNGEVSNIKPSLINVKDVENVTPNKALAEQINQADQTFRAQTAEVIIPNNTIDFKGERDDVRTRETNLGNAIADAMEAYGVKNFSKKTDFAVTNGGGIRASIAKGKVTRYDLISVLPFGNTIAQIDVKGSDVWTAFEHSLGAPTTQKDGKTVLTANGGLLHISDSIRVYYDINKPSGKRINAIQILNKETGKFENIDLKRVYHVTMNDFTASGGDGYSMFGGPREEGISLDQVLASYLKTANLAKYDTTEPQRMLLGKPAVSEQPAKGQQGSKGSKSGKDTQPIGDDKVMDPAKKPAPGKVVLLLAHRGTVSSGTEGSGRTIEGATVSSKSGKQLARMSVPKGSAHEKQLPKTGTNQSSSPEAMFVLLAGIGLIATVRRRKAS

SEQ ID NO: 2 - protein, 97 aa, Staphylococcus aureus
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQ

SEQ ID NO: 3 - protein, 104 aa, Staphylococcus aureus
MGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 4 - protein, 130 aa, Staphylococcus aureus
MNFNDIETMVKSKFKDIKKHAEEIAHEIEVRSGYLRKAEQYKRLEFNLSFALDDIESTAKDVQTAKSSANKDSVTVKGKAPNTLYIEKRNLMKQKLEMLGEDIDKNKESLQKAKEIAGEKASEYFNKAMN

SEQ ID NO: 5 - protein, 21 aa, Staphylococcus aureus
MGIIAGIIKVIKSLIEQFTGK

SEQ ID NO: 6 - protein, 21 aa, Staphylococcus aureus
MGIIAGIIKFIKGLIEKFTGK

SEQ ID NO: 7 - protein, 22 aa, Staphylococcus aureus
MEFVAKLFKFFKDLLGKFLGNN

SEQ ID NO: 8 - protein, 20 aa, Staphylococcus aureus
MAIVGTIIKIIKAIIDIFAK

SEQ ID NO: 9 - protein, 298 aa, Staphylococcus aureus
MNAIELSNVNYSSDQFNLKNISFKVPQGFVTGFIGRNGAGKTTIIRLIMDLYQPQTGVIRVLEEDMALNPIELKNRIGFVYSENYFNERWTTKQLEKMIAPFYRKWDHQVFEFYLEKFDLPINKSIKTFSTGMKMKLSLAVAFSHHAELYIFDEPTSGLDPLARNELLEIIQQELIDENKTIFMSTHIISDLEKIADYIIHLSDGEVILNGSKEQLLQRYQVVSGAIEDLDDELASLLIYEEHKRTGFIGLTEHAQVFKEILGHKVNITTPSIENLMVYLEKRKPKYHENIKLMEEGF

SEQ ID NO: 10 - protein, 226 aa, Staphylococcus aureus
MKALLIRNFKLRRYTLIIYVLLLTLYPFYIMLDSTKFFYLLQSFISPTILIIWILDAGHLFRLNRRLGGNDSYYFYMSLPVSKKQLLNANYITCIVLTLIGTLVISLYAYEADVIEPNSIYFSTAYAFVISNFLSIPIAFSQFTELRRVKVPYGIYVFTIIILVPFLFSIAIVLVNYFVLSQSSFPDLYSYILNIGFLIISIVILIVNYFKQLNKINTRKFKGGSR

SEQ ID NO: 11 - protein, 290 aa, Staphylococcus aureus
MKLEHITKKYGSNVVLNDIDFDFGDSRIVGLIGKNGVGKTTVMKVMNGNIIKFDGKVDIDNADNIGFLIEHPKLYDNKSGLYNLKLFAQVLGKGFDKAYTDKIIDAFGMRPYIKKKVKKYSMGMKQKLAIAVSLMNKPKFLILDEPTNGMDPDGSIDVLTTIKSLVNELDMRILISSHKLEDIELICDRAVFLRDGHFVQDVNMEEGVASDTTIVTVDHKDFDRTEKYLAEHFQLQNVDKADGHLMINAQKNYQVILKALSELDIYPKYIETRKSSLRDTYFNINQRGDK

SEQ ID NO: 12 - protein, 246 aa, Staphylococcus aureus
MRILNLVKYDFYSIFKSPLTYLAILVVSSLIATQSILMANSMDNPKHIIVYGSVFAAAKWLLLIIGLMFVVKTITRDFSQGTIQLYMSKVKTRVGYIISKTISIILISILFALIHYVILIVVQASSNGKNLAFSKYVDNLWFFLIFLLFFGLFLFLITLASQKTAMIFSLGVFLVLIVPFIKPFITFIPRYGEKVLDAFDYIPFAYLTDKMISSNFDFSNWQWVISLGSIVIFFILNILYVAKKDI